(19) United States
(12) Patent Application Publication
(10) Pub. No.: US 2025/0221941 A1
(43) Pub. Date: Jul. 10, 2025

(54) NEPHRON PROGENITOR EXOSOMES

(71) Applicants: VANDERBILT UNIVERSITY, Nashville, TN (US); The United States as Represented by the Department of Veterans Affairs, Washington, DC (US)

(72) Inventors: Lauren F. Woodard, Nashville, TN (US); Julie Bejoy, Nashville, TN (US)

(21) Appl. No.: 18/698,977

(22) PCT Filed: Oct. 6, 2022
(86) PCT No.: PCT/US2022/077692

§ 371 Date: Apr. 5, 2024

Related U.S. Application Data
(60) Provisional application No. 63/253,733, filed on Oct. 8, 2021.

(51) Int. Cl. A61K 9/50; A61P 13/12; C12N 5/00; C12N 5/071

(52) U.S. Cl. CPC A61K 9/5068; A61P 13/12; C12N 5/0018; C12N 5/0686; C12N 2506/02; C12N 2506/45; C12N 2510/00

(57) ABSTRACT

Disclosed herein are nephron progenitor extracellular vesicles (EVs) that can be used to treat acute kidney injury and methods of using same. For example, disclosed herein is a composition involving nephron progenitor extracellular vesicles (EVs), wherein the EVs are produced by a method that involves: engineering donor cells to express exogenous SNAI2, EYA1, and SIX1, thereby producing induced nephron progenitor (iNP) cells, or obtaining nephron progenitor-like cells from less differentiated cell types including pluripotent stem cells such as induced pluripotent stem cells or embryonic stem cells; culturing the iNP or NPC cells in a culture medium, thereby producing a conditioned medium; and collecting and purifying nephron progenitor EVs from the conditioned medium.

### CROSS-REFERENCE TO RELATED APPLICATIONS

This application claims benefit of U.S. Provisional Application No. 63/253,733, filed Oct. 8, 2021, which is hereby incorporated herein by reference in its entirety.

### SEQUENCE LISTING

This application contains a sequence listing filed in ST.26 format entitled “222230_2100_Sequence_Listing” created on Oct. 6, 2022. The content of the sequence listing is incorporated herein in its entirety.

### BACKGROUND

Of patients admitted to the intensive care unit, nearly one quarter develop acute kidney injury (AKI), which is associated with a substantial increase in mortality. Some patients with AKI suffer a lifelong loss of kidney function that is associated with a later diagnosis of chronic kidney disease (CKD) or end-stage kidney disease. The Kidney Disease Improving Global Outcomes (KDIGO) criteria for AKI is defined as either a rise in serum creatinine (sCr) exceeding either 0.3 mg/dl over 48 h or 1.5× baseline within the past 7 days, or urine production of less than 0.5 ml/kg/h maintained over 6 h (Lameire N H, et al. Lancet 2013 382:170-179). AKI patients often receive dialysis treatment which does not replace kidney function completely; dialysis is perhaps better thought of as renal supportive therapy instead or renal replacement therapy (Raina R, et al. Ther Apher Dial 2021 25:437-457). The AKI to CKD transition is associated with fibrosis and extracellular matrix deposition mediated by changes in integrin localization in the renal epithelium (Zuk A, et al. Am J Physiol 1998 275: C711-731). Administering a regenerative therapy soon after kidney injury may, in theory at least, lower mortality in patients with AKI and prevent the AKI to CKD transition. Stem cells have great promise as a future AKI treatment, but they have many practical difficulties including the high cost, infrastructure, batch variability, and delivery challenges to infuse the stem cells into the patient immediately after diagnosis (Pigeau G M, et al. Front Med (Lausanne) 2018 5:233).

### SUMMARY

Extracellular vesicles (EVs) are membrane-bound particles secreted from most cell types. The majority of EVs are small, less than 150 micrometer. Extracellular vesicles from different adult stem cells in various kidney injury models, including ischemia-reperfusion injury and cisplatin-treated models, have been demonstrated to have therapeutic effects. The EVs are enriched in microRNAs and proteins that modulate proliferation, angiogenesis, and apoptosis. In this way, the extracellular vesicles signal between cells of various cell types. In a number of systems, stem cell EVs have been shown to be regenerative.

Extracellular vesicles including exosomes provide many of the therapeutic benefits of stem cell therapies without the cost to derive patient-specific cells. EVs have many advantages over cell therapy strategies, including but not limited to: a lack of immunogenicity, greater ease of production, ability to store for off-the-shelf therapies, and the potential to add therapeutic RNA or protein payloads to the particles.

Nephron progenitor cells are stem cells that are potentially able to differentiate into all of the cell types of the adult nephron but are not present in the normal adult kidney, only the embryo. Three transcription factors (SNAI2, EYA1, and SIX1) are able to induce a nephron progenitor phenotype when expressed in human kidney tubular cells, resulting in induced nephron progenitor (iNP) cells. As disclosed herein, EVs produced by human nephron progenitor-like cells including these iNPs (NP-EVs) are to scale-up to produce a regenerative biologic therapy for acute kidney injury and to treat the ischemic injury that transplanted kidneys are subjected to.

Therefore, disclosed herein is a composition involving nephron progenitor extracellular vesicles (EVs), wherein the EVs are produced by a method that involves: engineering donor cells to express exogenous SNAI2, EYA1, and SIX1, thereby producing induced nephron progenitor (iNP) cells, or obtaining nephron progenitor-like cells from less differentiated cell types including pluripotent stem cells such as induced pluripotent stem cells or embryonic stem cells; culturing the iNP or NPC cells in a culture medium, thereby producing a conditioned medium; and collecting and purifying nephron progenitor EVs from the conditioned medium.

In some embodiments, the EVs have an average diameter of 50 nm to 150 nm, including 50, 60, 70, 80, 90, 100, 110, 120, 130, 140, or 150 nm.

In some embodiments, the EVs are loaded with a therapeutic agent. Therefore, in some embodiments, the composition further contains a pharmaceutically acceptable carrier. The therapeutic cargo may include any DNA. RNA, or protein expressed within the cells or loaded onto the EVs. In some embodiments, changes may be made to the EVs including but not limited to microRNA, modified nucleic acids, enzymes, antioxidants, antibodies, or nanobodies. In some embodiments, drugs including small molecules, lipids, and/or other biologic or artificial molecules may be added to the EVs at any stage of their production or purification or made to be removed from the EVs.

Also disclosed is a method for treating or preventing acute kidney injury in a subject that involves administering to the subject an effective amount of an EV composition disclosed herein. In some embodiments, the donor cells used to produce the EVs are autologous. However, in other embodiments, the donor cells are allogeneic.

In some embodiments, the EV composition is administered after onset of the injury. However, in other embodiments, the EV composition is administered prophylactically to a subject at risk for acute kidney injury.

The methods can in some embodiments be used to treat or prevent any form of kidney injury, disease, or trauma that can result in acute kidney injury. For example, in some embodiments, the subject is the recipient of a kidney transplant. In some embodiments, the subject is undergoing dialysis. In some embodiments, the subject has or is at risk of having kidney trauma, sepsis, emergent conditions, drug toxicity, alcohol toxicity, chemotherapy treatment, drug treatment, burns, dehydration, starvation, dieting, ketosis or eating disorders, herbal, diet, supplement, or environmental nephrotoxicity, rhabodomyolysis, amputation, cardiac bypass, cardiac arrest, premature birth, preeclampsia, viral infection including but not limited to HIV and COVID, lung disease, liver disease, heart disease, muscle disease, blood disease including sickle cell anemia, tumor lysis syndrome, diabetes, hypertension or high blood pressure, autoimmune disease including lupus, or any condition that could result in acute kidney injury, chronic kidney disease, nephron loss, or other nephropathy.

Also disclosed is a method that involves treating a donor kidney with effective amount of a disclosed EV composition prior to transplantation into a subject in need thereof.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B: HK2-C8 cells show evidence of reprogramming in vitro in NPSR media. (A) Protocol used for deriving iNPs from HK2 Clone 8 (HK2-C8) cells. (B) Live fluorescence imaging showing the increase in mCherry expression in the HK2-C8 cells during doxycycline-inducible reprogramming and transdifferentiation to iNPs.

FIGS. 2A to 2C: HK2 Clone 8 cells express nephron progenitor (NP) proteins at day 8 of reprogramming. (A) Brightfield images of HK2-C8 cells during the reprogramming (i) Day 0 (ii) Day 8. (B) Flow cytometry analysis showing expression of NP proteins SIX2 and CITED1 in the reprogrammed iNP cells. (C) Immunofluorescence of NP proteins SIX2, CITED1, PAX2 colocalized with RFP/mCherry in the reprogrammed cells.

FIG. 3. Isolation and characterization of iNP-derived extracellular vesicles (INP-EVs). (A) Immunofluorescence of exosome markers CD63, CD81, Alix and TSG101 in reprogrammed cells. (B) Protocol used for isolating extracellular vesicles enriched in exosomes from the nephron progenitor cell culture media. (C) Representative NTA histogram of the exosomes from Nanoparticle tracking analysis (NTA). (D) Presence of exosome-sized vesicles verified by electron microscopy (EM). (E) Proteomic analysis of EVs isolated from cell culture media at different stages of reprogramming.

FIG. 4. INP-EVs reduced cisplatin-induced apoptosis and increased proliferation in immortalized human tubule cells. Cisplatin treatment reduced proliferation (A) and viability (B) in HK2 cells. C. Ki-67 staining (i) and quantification (ii) with and without the iNP-EVs treatment. (D) Live Caspase 3/7 activity (i) and quantification (ii) analysis with and without the iNP-EVs treatment. Scale bar 100 μm **p<0.01, ***p<0.001, ****p≤0.0001.

FIG. 5. iNP-EVs reverse apoptosis of immortalized tubule cells caused by cell-free hemoglobin (CFH). (A) Schematic of the experimental design for a tissue culture model of septic injury to the renal tubular epithelium. (B) MTT activity (i) and LDH activity (ii) in HK2 cells with and without CFH treatment. (C) Cleaved Caspase 3 (CC3) staining (i) with and without CFH treatment and corresponding quantification (ii) (D) Ki-67 staining of the CFH treated group. (E) Lipid peroxidation by MDA assay with and without CFH treatment. (F) MTT assay analysis showing viability in the model with and without treatment with iNP-EVs. Scale bar: 100 μm. **p<0.01, ***p<0.001, ****p≤0.0001.

FIG. 6. INP-EVs treatment reduced the cisplatin-induced toxicity and cytokine release in kidney organoids. (A) Schematic of experimental design. (B) Schematic outline of the protocol used in differentiating iPSCs into kidney organoids on transwell. Brightfield image of the whole kidney organoids at day 25. (C) Immunostaining of the resulting organoid shows positive staining for distal tubule (ECAD) proximal tubule (LTL) and collecting duct (GATA3). Scale bar, 500 μm. (n=3). (D) Brightfield images of the organoids treated with cisplatin. (E) The impact of cisplatin on the (i) viability and cytotoxicity (ii) of cells within kidney organoids (n=3). (F) KIM-1 Elisa showing increased KIM-1 release in the cisplatin treated organoids than the control group. (n=3). (G) Co-staining of the iNP-EVs treated organoids with proliferation marker Ki-67 and kidney injury marker KIM-1 with distal tubule marker ECAD. (H) CBA assay analysis of conditioned media collected from cisplatin-treated organoids with or without treatment with iNP-EVs for cytokines IL6, IL1β, IL8 and IFN-γ. **p<0.01.

FIG. 7. Experimental setup to test iNP-EVs in a mouse model of ischemia reperfusion AKI.

FIG. 8. Effect of iNP-EVs on kidney injury biomarkers within 48 hours of treatment in I/R mice. (A) Graphical abstract of the experimental setup and sample collection time points. (B) The levels of serum BUN (i) and SCr (ii) were increased in both I/R group and I/R+EVs group 24 hours after surgery indicating successful injury. (C) Levels of urinary kidney injury markers KIM-1 (i) and NGAL (ii) were determined by ELISA.

FIG. 9. iNP-Evs reduced the injury marker expression as well as improved proliferation in tubule cells within 48 hours in I/R mice. (A) iNP-EV-treated mouse kidneys were stained for proximal tubule marker SLC3A1 (red) and kidney injury marker KIM-1 (green). (B) Immunostaining of the kidney tissue with costained with LTL for proximal tubules (green) and proliferative marker Ki-67 (red; positive cells noted with white arrows; i) and corresponding quantification of double positive cells (ii).

FIG. 10. Renal tissue histology after treatment with iNP-EVs. H&E (A) or PAS (B) stained kidney slices were performed for the three groups (sham, I/R, or I/R+iNP-EVs). Images shown are representative of each group. Scale bar 700 μm.

FIG. 11. Comparison of kidney injury markers, survival, and transglomerular filtration rate (tGFR) following ischemia reperfusion acute kidney injury with or without iNP-EV treatment. (A) Graphical abstract of the experimental set up and sample collection time points. The levels of serum BUN (B) and SCr (C) at week 0 before iNP-EV administration and weeks 1 and 2 following surgery and iNP-EVs. (D) The ELISA results showing levels of kidney injury marker NGAL in urine for each group. n=10 (E) Survival Kaplan Meier curves to compare mortality over two weeks following I/R or sham surgery. (F) tGFR measurements of mice taken at 2 weeks after I/R or sham surgery.

FIG. 12. Renal tissue morphology with or without treatment with iNP-EVs. (A) Lower magnification (upper panel) and higher magnification (lower panel) immunohistochemistry images of PAS staining. (B) Blind scoring of tubular dilation (i) and Cast (ii) formation. n=3.
magnification (upper panel) and higher magnification (lower panel) immunohistochemistry images of Sirius red staining. Fibrotic areas are indicated by arrows. (B) Blinded scoring of Sirius red in three groups. n=3.

FIG. 14. Effects of iNP-EVs on high-glucose treated podocytes. (A) Schematic of experimental design. (B) Cleaved Caspase 3 (CC3, green) co-staining with actin cytoskeleton (F-Actin, red) and DAPI (blue). (C) Ki-67 staining (red) with podocyte marker PODXL (green). n=3 (D) Flow cytometry analysis of apoptosis by using Annexin V (FITC) to stain for inversion of the cell membrane and propidium iodide as a proxy for membrane permeability. Scale bar 100 μm. n=3.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “progenitor cell” refers to a cell which is capable of differentiating along one or a plurality of developmental pathways, with or without self-renewal. Progenitor cells may be pluripotent, multipotent, oligopotent or unipotent. Typically, progenitor cells are unipotent or oligopotent and are capable of at least limited self-renewal.

The term “nephron progenitor cells” refers to progenitor cells that have “nephron progenitor activity” and can differentiate into some or all nephron segments (other than collecting duct) which include nephron epithelia such as connecting segment, distal convoluted tubule (DCT) cells, distal straight tubule (DST) cells, proximal straight tubule (PST) segments 1 and 2 PST cells, podocytes, glomerular endothelial cells, ascending Loop of Henle and/or descending Loop of Henle, although without limitation thereto. Nephron progenitor cells are also capable of self-renewal under permissive conditions.

The terms “induced nephron progenitors” and “induced nephron progenitor cells” (iNPs) refer to cells which do not normally have nephron progenitor potential or activity, or have minimal, insubstantial or insufficient nephron progenitor potential or activity, but are induced to have nephron progenitor activity as a result of expression of the SNAI2, EYA1 and SIX1 genes disclosed herein. The term “nephron progenitor-like cells” or NPCs refer to cells which have been incubated with specific media to differentiate them from a starting cell type such as pluripotent stem cell (induced pluripotent stem cell or embryonic stem cell) into a cell resembling a nephron progenitor and/or having nephron progenitor properties.

The terms “differentiate”, “differentiating” and “differentiated”, relate to progression of a cell from an earlier or initial stage of a developmental pathway to a later or more mature stage of the developmental pathway. It will be appreciated that in this context “differentiated” does not mean or imply that the cell is fully differentiated and has lost pluripotentiality or capacity to further progress along the developmental pathway or along other developmental pathways. Differentiation may be accompanied by cell division.

As will be well understood in the art, the stage or state of differentiation of a cell may be characterized by the expression and/or non-expression of one of a plurality of markers. In this context, by “markers” is meant nucleic acids or proteins that are encoded by the genome of a cell, cell population, lineage, compartment or subset, whose expression or pattern of expression changes throughout development. Nucleic acid marker expression may be detected or measured by any technique known in the art including nucleic acid sequence amplification (e.g. polymerase chain reaction) and nucleic acid hybridization (e.g. microarrays, Northern hybridization, in situ hybridization), although without limitation thereto. Protein marker expression may be detected or measured by any technique known in the art including flow cytometry, immunohistochemistry, immunoblotting, protein arrays, protein profiling (e.g 2D gel electrophoresis), although without limitation thereto.

It will be appreciated that particular aspects of the invention relate to the induction of nephron progenitor activity, or at least nephron progenitor potential, in a cell, or a tissue or organ comprising the cell, by the expression of a SNAI2 gene, an EYA1 gene and a SIX1 gene in the cell, tissue or organ that does not normally have nephron progenitor activity as described above. In this context, the cell has no ability, minimal, insubstantial or insufficient ability to differentiate into some or all nephron segments as described above. This may be any cell that can be propagated in vitro or in vivo and in which a SNAI2 gene, an EYA1 gene and a SIX1 gene are not normally expressed, or are expressed at a level which does not induce or otherwise cause said cell to have nephron progenitor activity.

In one embodiment, the cell is a differentiated cell or cell line which does not normally have nephron progenitor activity as described above. Differentiated cells have no or minimal or substantial intrinsic progenitor potential and so are a “safe” choice for producing nephron progenitors because there is less chance that these cells may become tumorigenic following expression of a SNAI2 gene, an EYA1 gene and a SIX1 gene. Non-limiting examples include fibroblasts, renal cells such as adult renal epithelial cells (e.g HK2 cells, hRPTECs), although without limitation thereto.

The term “nucleic acid” refers to single- or double-stranded DNA and RNA. DNA includes genomic DNA and cDNA. RNA includes mRNA, RNA, RNAi, siRNA, CRNA, guide RNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

The term “gene” refers to a structural unit or region of a genome that comprises a nucleotide sequence encoding an amino acid sequence of a protein. Typically, a gene comprises one or more exons that encode a protein and non-coding genetic elements such as one or more introns, 5′ and 3′ UTR and regulatory regions such as a promoter and/or enhancer.

The term “protein” refers to an amino acid polymer. The amino acids may be natural or non-natural amino acids, D- or L-amino acids as are well understood in the art. The term “protein” includes and encompasses “peptide”, which is typically used to describe a protein having no more than fifty (50) amino acids and “polypeptide”, which is typically used to describe a protein having more than fifty (50) amino acids.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

### Induced Nephron Progenitor Cells

Disclosed herein are extracellular vesicles derived from induced nephron progenitor (iNP) cells. Methods of making iNP cells are described in US2020/00157572A1, which is incorporated by reference in its entirety for the teaching of these compositions and methods. In particular, a cell can be induced to possess nephron progenitor potential or nephron progenitor activity by expressing at least one exogenous nucleic acid that comprises a nucleotide sequence of a SNAI2 gene, an EYA1 gene and a SIX1 gene, or respective fragments thereof, at a level that induces said cell to have nephron progenitor potential or activity.

In some embodiments, the starting cell does not normally have nephron progenitor activity. In this context, the cell has no ability, minimal, insubstantial or insufficient ability to differentiate into some or all nephron segments. This may be any cell that can be propagated in vitro or in vivo and in which a SNAI2 gene, an EYA1 gene and a SIX1 gene are not normally expressed, or are expressed at a level which does not induce or otherwise cause said cell to have nephron progenitor activity.

In some embodiments, the cell is a differentiated cell or cell line which does not normally have nephron progenitor activity. Differentiated cells have no or minimal or substantial intrinsic progenitor potential and so are a “safe” choice for producing nephron progenitors because there is less chance that these cells may become tumorigenic following expression of a SNAI2 gene, an EYA1 gene and a SIX1 gene. Non-limiting examples include fibroblasts, renal cells such as adult renal epithelial cells (e.g HK2 cells, hRPTECs).

In some embodiments, the cell may be a progenitor cell in its normal state. A progenitor cell may have the advantage that it is multipotential and, as a result of expressing respective nucleotide sequences of a SNAI2 gene, an EYA1 gene and a SIX1 gene, or fragments thereof, is not only capable of differentiating into a nephron but also into one or more other renal cell types, renal structures or tissues (e.g. ureteric components such as collecting ducts or renal vasculature) as a result of its multipotentiality. Non-limiting examples of progenitor cells include urine-derived stem cells, glomerular parietal epithelial cells, ureteric epithelial progenitor cells, intermediate mesoderm and posterior primitive streak cells, without limitation thereto. The in vitro production of renal progenitor cells (such as from human embryonic stem cells or iPSCs) that may be used according to the invention is described in detail in International Publications WO2014/197934 and WO2016/94948, which are incorporated by reference for these teachings.

In some embodiments, the cell may be a primary postnatal cell type with no renal identity (e.g. fibroblast) or other mature adult cell type with no progenitor activity (e.g. primary proximal tubule epithelial cells).

In some embodiments, the cell may be a pluripotent stem cell. In this embodiment, the SNAI2, EYA1 and SIX1 genes would be inducing nephron progenitor status rather than growth factor induction.

In some embodiments, the cell is autologous patient-derived, heterologous haplotype-matched or heterologous so to reduce or avoid the generation of an immune response in a patient to whom the exosomes are delivered.

In some embodiments, the cell is induced to possess nephron progenitor potential or nephron progenitor activity by expressing at least one exogenous nucleic acid that comprises a nucleotide sequence of a SNAI2 gene, an EYA1 gene and a SIX1 gene, or respective fragments thereof, at a level that induces said cell to have nephron progenitor potential or activity.

In some embodiments, human SNAI2 gene comprises
the nucleotide sequence:
(hSNAI2, NM_003068.4, SEQ ID NO: 1)
ATGCCGCGCTCCTTCCTGGTCAAGAAGCATTTCAACGCCTCCAAAAAGCC
AAACTACAGCGAACTGGACACACATACAGTGATTATTTCCCCGTATCTCT
ATGAGAGTTACTCCATGCCTGTCATACCACAACCAGAGATCCTCAGCTCA
GGAGCATACAGCCCCATCACTGTGTGGACTACCGCTGCTCCATTCCACGC
CCAGCTACCCAATGGCCTCTCTCCTCTTTCCGGATACTCCTCATCTTTGG
GGCGAGTGAGTCCCCCTCCTCCATCTGACACCTCCTCCAAGGACCACAGT
GGCTCAGAAAGCCCCATTAGTGATGAAGAGGAAAGACTACAGTCCAAGCT
TTCAGACCCCCATGCCATTGAAGCTGAAAAGTTTCAGTGCAATTTATGCA
ATAAGACCTATTCAACTTTTTCTGGGCTGGCCAAACATAAGCAGCTGCAC
TGCGATGCCCAGTCTAGAAAATCTTTCAGCTGTAAATACTGTGACAAGGA
ATATGTGAGCCTGGGCGCCCTGAAGATGCATATTCGGACCCACACATTAC
CTTGTGTTTGCAAGATCTGCGGCAAGGCGTTTTCCAGACCCTGGTTGCTT
CAAGGACACATTAGAACTCACACGGGGGAGAAGCCTTTTTCTTGCCCTCA
CTGCAACAGAGCATTTGCAGACAGGTCAAATCTGAGGGCTCATCTGCAGA
CCCATTCTGATGTAAAGAAATACCAGTGCAAAAACTGCTCCAAAACCTTC
TCCAGAATGTCTCTCCTGCACAAACATGAGGAATCTGGCTGCTGTGTAGC
ACACTGA.
In some embodiments, human SIX1 gene comprises
the nucleotide sequence:
(hSIX1, NM_005982.3, SEQ ID NO: 2)
ATGTCGATGCTGCCGTCGTTTGGCTTTACGCAGGAGCAAGTGGCGTGCGT
GTGCGAGGTTCTGCAGCAAGGCGGAAACCTGGAGCGCCTGGGCAGGTTCC
TGTGGTCACTGCCCGCCTGCGACCACCTGCACAAGAACGAGAGCGTACTC
AAGGCCAAGGCGGTGGTCGCCTTCCACCGCGGCAACTTCCGTGAGCTCTA
CAAGATCCTGGAGAGCCACCAGTTCTCGCCTCACAACCACCCCAAACTGC
AGCAACTGTGGCTGAAGGCGCATTACGTGGAGGCCGAGAAGCTGCGCGGC
CGACCCCTGGGCGCCGTGGGCAAATATCGGGTGCGCCGAAAATTTCCACT
GCCGCGCACCATCTGGGACGGCGAGGAGACCAGCTACTGCTTCAAGGAGA
AGTCGAGGGGTGTCCTGCGGGAGTGGTACGCGCACAATCCCTACCCATCG
CCGCGTGAGAAGCGGGAGCTGGCCGAGGCCACCGGCCTCACCACCACCCA
GGTCAGCAACTGGTTTAAGAACCGGAGGCAAAGAGACCGGGCCGCGGAGG
CCAAGGAAAGGGAGAACACCGAAAACAATAACTCCTCCTCCAACAAGCAG
AACCAACTCTCTCCTCTGGAAGGGGGCAAGCCGCTCATGTCCAGCTCAGA
AGAGGAATTCTCACCTCCCCAAAGTCCAGACCAGAACTCGGTCCTTCTGC
TGCAGGGCAATATGGGCCACGCCAGGAGCTCAAACTATTCTCTCCCGGGC
TTAACAGCCTCGCAGCCCAGTCACGGCCTGCAGACCCACCAGCATCAGCT
CCAAGACTCTCTGCTCGGCCCCCTCACCTCCAGTCTGGTGGACTTGGGGT
CCTAA.
In some embodiments, human EYA1 gene comprises
the nucleotide sequence:
(hEYA1, NM_172058.2, SEQ ID NO: 3)
ATGGAAATGCAGGATCTAACCAGCCCGCATAGCCGTCTGAGTGGTAGTAG
TGAATCCCCCAGTGGCCCCAAACTCGGTAACTCTCATATAAATAGTAATT
CCATGACTCCCAATGGCACCGAAGTTAAAACAGAGCCAATGAGCAGCAGT
GAAACAGCTTCAACGACAGCCGACGGGTCTTTAAACAATTTCTCAGGTTC
AGCAATTGGGAGCAGTAGTTTCAGCCCACGACCAACTCACCAGTTCTCTC
CACCACAGATTTACCCTTCCAACAGACCATACCCACATATTCTCCCTACC
CCTTCCTCACAAACTATGGCTGCATATGGGCAAACACAGTTTACCACAGG
AATGCAACAAGCTACAGCCTATGCCACGTACCCACAGCCAGGACAGCCGT
ACGGCATTTCCTCATATGGTGCATTGTGGGCAGGCATCAAGACTGAAGGT
GGATTGTCACAGTCTCAGTCACCTGGACAGACAGGATTTCTCAGCTATGG
CACAAGCTTCAGTACCCCTCAACCTGGACAGGCACCATACAGCTACCAGA
TGCAAGGTAGCAGTTTTACAACATCATCAGGAATATATACAGGAAATAAT
TCACTCACAAATTCCTCTGGATTTAATAGTTCACAGCAGGACTATCCGTC
TTATCCCAGTTTTGGCCAGGGTCAGTACGCACAGTATTATAACAGCTCAC
CGTATCCAGCACATTATATGACCAGCAGCAACACCAGCCCAACGACACCA
TCCACCAATGCCACTTACCAGCTTCAAGAACCGCCATCTGGCATCACCAG
CCAAGCAGTTACAGATCCCACAGCAGAGTACAGCACAATCCACAGCCCAT
CAACACCCATTAAAGATTCAGATTCTGATCGATTGCGTCGAGGTTCAGAT
GGGAAATCACGTGGACGGGGCCGAAGAAACAATAATCCTTCACCTCCCCC
AGATTCTGATCTTGAGAGAGTGTTCATCTGGGACTTGGATGAGACAATCA
TTGTTTTCCACTCCTTGCTTACTGGGTCCTACGCCAACAGATATGGGAGG
GATCCACCCACTTCAGTTTCCCTTGGACTGCGAATGGAAGAAATGATTTT
CAACTTGGCAGACACACATTTATTTTTTAATGACTTAGAAGAATGTGACC
AAGTCCATATAGATGATGTTTCTTCAGATGATAACGGACAGGACCTAAGC
ACATATAACTTTGGAACAGATGGCTTTCCTGCTGCAGCAACCAGTGCTAA
CTTATGTTTGGCAACTGGTGTACGGGGCGGTGTGGACTGGATGAGAAAGT
TGGCCTTCCGCTACAGACGGGTAAAAGAGATCTACAACACCTACAAAAAT
AATGTTGGAGGTCTGCTTGGTCCAGCTAAGAGGGAAGCCTGGCTGCAGTT
GAGGGCCGAAATTGAAGCCCTGACCGACTCCTGGTTGACACTGGCCCTGA
AAGCACTCTCGCTCATTCACTCCCGGACAAACTGTGTGAATATTTTAGTA
ACAACTACTCAGCTCATCCCAGCATTGGCGAAAGTCCTGCTGTATGGGTT
AGGAATTGTATTTCCAATAGAAAATATTTACAGTGCAACTAAAATAGGAA
AAGAAAGCTGTTTTGAGAGAATAATTCAAAGGTTTGGAAGAAAAGTGGTG
TATGTTGTTATAGGAGATGGTGTAGAAGAAGAACAAGGAGCAAAAAAGCA
CGCGATGCCCTTCTGGAGGATCTCCAGCCACTCGGACCTCATGGCCCTGC
ACCATGCCTTGGAACTGGAGTACCTGTAA.

In order to facilitate delivery of the at least one exogenous nucleic acid (such as comprising the EYA1, SIX1 and SNAI2 nucleotide sequences set forth in SEQ ID NOs.: 1-3, or variants or fragments thereof) to a cell, the at least one exogenous nucleic acid can be present in a genetic construct. Suitably, the genetic construct is an “expression construct” wherein the at least one exogenous nucleic acid is operably linked or operably connected to one or more regulatory nucleotide sequences that control, facilitate or regulate expression of the EYA1, SIX1 and SNAI2 nucleotide sequences. By “operably linked” is meant that said additional nucleotide sequence(s) is/are positioned relative to the EYA1, SIX1 and SNAI2 nucleotide sequences preferably to initiate, regulate or otherwise control transcription of the EYA1, SIX1 and SNAI2 nucleotide sequences. Typically, said one or more regulatory nucleotide sequences may include, but are not limited to, promoter sequences, leader or signal sequences, ribosomal binding sites, polyadenylation sequences, transcriptional start and termination sequences, donor/acceptor splice sites, Kozak and translational start and termination sequences, and/or enhancer or activator sequences. The choice of said one or more regulatory nucleotide sequences may be at least partly dependent on the host cell type used for expression, particularly according to the origin of the host cell (e.g. mammalian or other vertebrates, plant, bacterial, yeast etc). Suitably, the one or more regulatory nucleotide sequences are operable in a human or other mammalian cell.

Broadly, the genetic construct may be in the form of, or comprise genetic components of, a plasmid, a transposon, a bacteriophage, a cosmid, a yeast or bacterial artificial chromosome as are well understood in the art. The genetic construct may be either a self-replicating extra-chromosomal construct such as a plasmid, or more preferably a construct that integrates into a host cell genome. In some embodiments, the genetic construct is a “non-viral” genetic construct. By this is meant that the genetic construct does not comprise, or is substantially free of, genetic elements and/or nucleotide sequences of viral origin. In the particular context of humans, the genetic construct does not comprise, or is substantially free of, genetic elements and/or nucleotide sequences of viral vectors typically used in human gene therapy such as lentivirus, adenovirus, poxvirus (e.g vaccinia virus) and/or retrovirus vectors, although without limitation thereto.

In some embodiments, the genetic construct is a plasmid that comprises one or more components of a transposon. The one or more components of the transposon suitably include inverted repeat (IR) sequences positioned at 5′ and 3′ terminus of the transposon.

The genetic construct may further comprise a nucleotide sequence encoding a transposase which facilitates insertion of the transposon-containing elements of the genetic construct into a host cell genome. Typically, the transposase nucleotide sequence would be located 5′ of 5′ IR or 3′ of 3′ IR. Alternatively, the transposase is encoded by a nucleotide sequence of a separate plasmid. An example of a transposon-containing genetic construct is a “piggyBac” construct. In this regard, reference is made to Woodard & Wilson, 2015, Trends. Biotechnol. 33 525 which provides a review of “piggyBac” expression constructs and transposase systems for genomic insertion.

In some embodiments, the genetic construct comprises respective nucleotide sequences of a SNAI2 gene, an EYA1 gene and a SIX1 gene, or respective fragments thereof located in the same genetic construct. It will be appreciated that an advantage of the nucleotide sequences of the SNAI2 gene, the EYA1 gene and the SIX1 gene, or respective fragments being located in the same genetic construct is that this facilitates co-ordinated expression of the SNAI2 gene, the EYA1 gene and the SIX1 gene, and/or their respective encoded proteins. Co-ordinated expression of the SNAI2 gene, the EYA1 gene and the SIX1 gene, and/or their respective encoded proteins may facilitate at least comparable, or preferably stoichiometric expression of the SNAI2 gene, the EYA1 gene and the SIX1 gene, and/or their respective encoded proteins in a cell. It will be appreciated that the term “stoichiometric” in this context does not mean or imply 1:1:1 expression (i.e on a molecule-for-molecule basis), but can include tolerable variation of no more than 10-fold, 9-fold, 8-fold, 7-fold, 6-fold, 5-fold, 4-fold, 3-fold or 2-fold between any two of the SNAI2 gene, the EYA1 gene and the SIX1 gene, and/or their respective encoded proteins.

In some embodiments, each of the respective nucleotide sequences are separated by intervening nucleotide sequences. In some embodiments, the intervening nucleotide sequences may encode one or more same or different peptides that facilitate post-translational cleavage and separation of the protein products of the expressed SNAI2, EYA1 and a SIX1 nucleotide sequences. Non-limiting examples of suitable peptides include self-cleaving peptides such as 2A peptides and/or variants of these.

It will be appreciated that other genetic constructs may be suitable, inclusive of transient expression constructs without permanent or stable integration of the SNAI2, EYA1 and SIX1 nucleic acids into the genome.

Suitably, the genetic construct comprises one or more same or different promoters. In some embodiments, the genetic construct comprises a single promoter that is operably linked to the respective nucleotide sequences of a SNAI2 gene, an EYA1 gene and a SIX1 gene, or respective fragments thereof, located in the same genetic construct. The promoter may be a constitutive or inducible promoter, preferably operable in a human or other mammalian cell. Suitably, the promoter is inducible, repressible or otherwise regulatable. Non-limiting examples of such promoters are antibiotic-inducible or repressible promoters (e.g doxycycline, tetracycline inducible or repressible promoters), alcohol-inducible promoters, steroid-inducible promoters and metal-inducible promoters, although without limitation thereto. In one embodiment, the promoter is a doxycycline-inducible promoter. A particular example of a doxycycline-inducible promoter includes a tetracycline-responsive element (TRE).

### Extracellular Vehicles (EVs)

Disclosed herein are extracellular vesicles (EVs) that can be secreted by an iNP cell. Cells secrete EVs with a broad range of diameters and functions, including apoptotic bodies (1-5 μm), microvesicles (100-1000 nm in size), and vesicles of endosomal origin, known as exosomes (50-150 nm). Also disclosed is a method for making the disclosed EVs that involves culturing the disclosed iNP cells. The method can further involve purifying EVs from the cells. EVs produced from cells can be collected from the culture medium by any suitable method. Typically a preparation of EVs can be prepared from cell culture or tissue supernatant by centrifugation, filtration or combinations of these methods. For example, EVs can be prepared by differential centrifugation, that is low speed (<20000 g) centrifugation to pellet larger particles followed by high speed (>100000 g) centrifugation to pellet EVs, size filtration with appropriate filters, gradient ultracentrifugation (for example, with sucrose gradient) or a combination of these methods.

The disclosed extracellular vesicles further may be loaded with a therapeutic agent. Suitable therapeutic agents include but are not limited to therapeutic drugs (e.g., small molecule drugs), therapeutic proteins, and therapeutic nucleic acids (e.g., therapeutic RNA). In some embodiments, the disclosed extracellular vesicles comprise a therapeutic RNA (also referred to herein as a “cargo RNA”). In some embodiments, the cargo RNA is an miRNA, shRNA, mRNA, ncRNA, sgRNA or any combination thereof.

In some embodiments, the therapeutic cargo is a membrane-permeable pharmacological compound that is loaded into the EV after it is secreted by the cell. In some embodiments, the cargo is an anti-cancer agent that can cause apoptosis or pyroptosis of a targeted tumor cell.

To achieve loading of small RNAs into EVs, transfection-based approaches have been proposed. Other reports have shown that using vector-induced expression of small RNAs in cells, small RNA loading into EVs can be achieved. Alternatively, EV donor cells may be transfected with small RNAs directly. Incubation of tumor cells with chemotherapeutic drugs is also another method to package drugs into EVs. To stimulate formation of drug-loaded EVs, cells are irradiated with ultraviolet light to induce apoptosis. Alternative approaches such as fusogenic liposomes also leads loading drugs into EVs.

In some embodiments, the therapeutic cargo is loaded into the EVs by diffusion via a concentration gradient.

### Methods

Also contemplated herein are methods for using the disclosed EVs. For example, the disclosed extracellular vesicles may be used to treat for acute kidney injury, kidney damage or disease in a subject, organ transplant, or otherwise improve cardiorenal outcomes.

The disclosed EVs may be administered to a subject or transplant organ by any suitable means. A transplant organ may be kept in fluid containing EVs or EVs may be administered to the organ during harvest or transplantation via injection or infusion of the parenchyma, ureter, ducts, or vessels. Administration to a human or animal subject may be selected from parenteral, intramuscular, intracerebral, intravascular, subcutaneous, parenchymal, or transdermal administration. Typically the method of delivery is by injection. Catherization of the ureter and retrograde infusion of the kidney area may also be used. Preferably the injection is intravascular (e.g. intravenous). A physician will be able to determine the required route of administration for each particular patient.

The EVs are preferably delivered as a composition. The composition may be formulated for parenteral, intramuscular, intracerebral, intravascular (including intravenous), subcutaneous, parenchymal, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs. EVs may be embedded in a slow-release formulation such as a hydrogel.

Parenteral administration is generally characterized by injection, such as subcutaneously, intramuscularly, or intravenously. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone.

Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration should be sterile, as is known and practiced in the art.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection.

Preferably, the dose of a single intramuscular injection is in the range of about 5 to 20 μg. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Extracellular Vesicles from Induced Nephron Progenitor Cells Reduce Urinary NGAL in Mice with Ischemia/Reperfusion Kidney Injury

### Introduction

Given that much of the effect of administered stem cells is paracrine, factors or biological products such as extracellular vesicles (EVs) or other products derived from these cells may exert similar effects. EVs or exosomes have practical advantages over live cell products for treatment of time-sensitive conditions such as AKI, including but not limited to: a lack of immunogenicity, greater ease of production, ability to store for off-the-shelf therapies, and the potential to add therapeutic RNA or protein payloads to the particles. The source of EVs is expected to affect payload including protein, RNA, and lipid components. For this reason, it was hypothesized that kidney-derived stem cell exosomes may be beneficial in a mouse model of ischemia/reperfusion (I/R) injury. For example, exosomes from urine-derived stem cells were found to reduce the urine volume and urinary microalbumin excretion in a diabetic rat model (Jiang, Z. Z., et al. Stem Cell Res Ther 2016 7:24; Chen, C. Y., et al. Theranostics 2018 8:1607-1623).

In this study, EVs were derived from induced nephron progenitor-like cells (INPs). Native nephron progenitor cells are required for development of the mammalian kidney as they participate in mutual signaling processes with the developing ureteric bud, self-renew, and differentiate into the nephron segments except collecting duct (Saxen, L. & Sariola, H. Pediatr Nephrol 1987 1:385-392). Human nephron progenitor cells can be isolated from human embryos but not from adult kidneys because nephrogenesis ceases shortly before full term birth (Ryan, D., et al. EBioMedicine 2018 27:275-283). Therefore, non-native sources of nephron progenitors present a more practical option for derivation of EVs. One potential source are nephron progenitors derived from human pluripotent stem cells (Takasato, M., et al. Nat Cell Biol 2014 16:118-126; Brown, A. C., et al. Dev Cell 2015 34:229-241; Li, Z., et al. Cell Stem Cell 2016 19:516-529). However, pluripotent stem cell maintenance is time-consuming and expensive. In contrast, iNPs from a clone of HK-2 cells are reproducibly obtained in 8 days, requiring only three days of culture in nephron progenitor media (Vanslambrouck, J. M. et al. Kidney Int. 2019 95 (5): 1153-1166). The reprogrammed iNPs are sensitive to the type of media in which they are cultured and have optimized this parameter (Tanigawa, S., et al. Cell Rep 2016 15:801-813). Three media types were tested: CDBLY (Tanigawa, S., et al. Cell Rep 2016 15:801-813), NPEM (Brown, A. C., et al. Dev Cell 2015 34:229-241), and NPSR (Li, Z., et al. Cell Stem Cell 2016 19:516-529). Induction of nephron progenitor marker genes was highest in NPSR, providing a critical element for expansion of the iNPs (Li, Z., et al. Cell Stem Cell 2016 19:516-529; Tanigawa, S., et al. Cell Rep 2016 15:801-813). The reprogrammed HK-2-derived iNPs were shown to contribute to the nephron progenitor compartment in a cell recombination assay (Hendry, C. E., et al. J Am Soc Nephrol 2013 24:424-1434).

Exosomes purified from other stem cell types show great therapeutic promise for kidney regeneration following injury. EVs from nephron progenitors have yet to be tested, possibly due to the lack of a population of cells that can be easily purified from a native source. It was hypothesized that induced nephron progenitor-like cell-derived extracellular vesicles (iNP-EVs) would have therapeutic efficacy in a mouse model of ischemia/reperfusion (I/R) AKI. Although exactly how closely renal repair recapitulates developmental pathways is debatable, there are many shared mechanisms that suggest that renal progenitor cells could produce exosomes to exert beneficial paracrine effects (Little, M. H. & Kairath, P. J Am Soc Nephrol 2017 28:34-46). Other studies have evaluated nephron progenitors (Toyohara, T., et al. Stem Cells Transl Med 2015 4:980-992; Aggarwal, S., et al. Sci Rep 2016 6:37270) or extracellular vesicles/exosomes (Herrera Sanchez, M. B., et al. Stem Cell Res Ther 2014 5:124; Burger, D., et al. Am J Pathol 2015 185:2309-2323; Ren, Y., et al. Stem Cell Res Ther 2020 11:410; Yu, L., et al. Stem Cell Res Ther 2021 12:379) for the treatment of acute kidney injury with promising results, but there are no studies as of yet on NP-derived EVs (Grange, C. & Bussolati, B. Nature Reviews Nephrology 2022 18:499-513). NPs are a kidney-specific self-renewing cell type, so there are likely to be kidney-specific factors that are not present in EVs derived from alternative cell types. The EVs were purified with polyethylene glycol (PEG) because this method does not require lengthy ultracentrifugation steps, making it more clinically relevant. In vitro tissue culture models of nephrotoxic and septic AKI were then used to analyze the protective effect of EVs. Improved cell viability and reduced cytokine release when these models of AKI were treated with iNP-EVs. Focus was given to one ambitious treatment timepoint of administration of the iNP-EVs at 24 h post-AKI. This late stage was chosen rather than a prevention timepoint in order to assess the potential for translation of this study as the majority of patients with AKI present as such many hours after the insult has taken place (de Caestecker, M., et al. J Am Soc Nephrol 2015 26:2905-2916). Future studies with the iNP-EVs will explore other timepoints of administration, dosing, as well as how applicable the findings of this study may be across various mouse models of AKI (Skrypnyk, N. I., et al. Am J Physiol Renal Physiol 2016 310: F972-984). Finally, iNP-EVs were tested in a model of diabetic kidney disease using podocytes derived from human induced pluripotent stem cells that were treated with high-glucose and found possible improvement in the actin cytoskeleton of the iNP-EV treated podocytes.

### Methods

### Induced Nephron Progenitor Cells

iNPs were generated from HK2-C8 using our previously published method of doxycycline-inducible expression of the pT-SES factors SNAI2, EYA1, and SIX1 from integrated piggyBac transposons in the clonal HK-2 derivative cell line Clone 8 (HK2-C8) (Vanslambrouck, J. M. et al. Kidney Int. 2019 95 (5): 1153-1166). Briefly, HK2-C8 cells were maintained in HK2 growth media (HK2GM) consisting of DMEM/F-12, HEPES (Gibco, Amarillo, TX) supplemented with 5 μg/mL insulin, 5 μg/mL transferrin and 5 ng/ml sodium selenite solution (ITS; Sigma Aldrich, St. Louis, MO), 100 U/mL penicillin and 100 g/mL streptomycin solution (PenStrep; Gibco), 0.1 μM hydrocortisone (Sigma Aldrich), 2 nmol/L L-glutamine (GlutaMAX; Gibco) and 10% fetal bovine serum. To generate iNPs from the HK2-C8 cell line, 60-80% confluent HK2-C8 cells were treated with HK2GM containing 2 mM valproic acid (VPA; Sigma Aldrich) and 2 mg/ml doxycycline (Sigma Aldrich) for 2 days, replaced with HK2GM media containing 2 mg/ml doxycycline for initiate the reprogramming for next 3 days, followed by changing the media to nephron progenitor media (NPSR) containing 2 mg/ml doxycycline for the last 3 days of reprogramming. During this procedure, the media was changed every day and saved for exosome production during the last 3 days. The NPSR media consists of DMEM/F-12, HEPES with 1×GlutaMAX, 1×non-essential amino acids (NEAA; Fisher Scientific, Waltham, MA), 0.1 μM 2-Mercaptoethanol (Fisher Scientific), 1×PenStrep (Corning, Corning, NY), 1×B-27 supplement, vitamin A (Fisher Scientific), 1×ITS (Fisher Scientific), 50 ng/ml BMP7 (Fisher Scientific), 200 ng/ml FGF2 (Peprotech, Rocky Hill, NJ), Heparin (Sigma), 10 UM Y27632 (Stemcell Technologies, Vancouver, BC), 10 ng/ml Leukemia Inhibitory Factor human (LIF; EMD Millipore, Darmstadt, Germany), 1 μM CHIR99021 (ReagentsDirect, Encinitas, CA), 50 nM LDN193189 (ReagentsDirect), and 0.2 μM A83-01 (APExBIO Technology, Houston, TX).

Immunocytochemistry The cells were washed with phosphate-buffered saline (PBS) and fixed for 15 min in 4% paraformaldehyde (PFA; Electron Microscopy Sciences, Hatfield, PA) in PBS. For intracellular markers, the cells were then permeabilized with 0.1% Triton X-100 (Sigma Aldrich). The samples were blocked with 5% FBS in PBS for 30 min and incubated with appropriate dilutions of primary antibodies for mCherry, SIX2, RFP, PAX2 or CITED1 (Table 2) for 4 h at room temperature or overnight at 4° C. The cells were then washed and incubated with the corresponding secondary antibodies at room temperature for 1 h (Table 2). The samples were counterstained with DAPI (1:1000) for nuclear staining and visualized using a ZOE fluorescent microscope.

### Extracellular Vesicle Isolation

HK2-iNPs were immunostained with exosome-specific markers such as CD63, CD81, Alix and TSG101. EVs were isolated from culture medium using an established method (Rider, M. A., et al. Sci Rep. 2016 6:23978). Conditioned cell culture medium containing the vesicles was centrifuged at 500×g for 5 min to remove the dead cells. The supernatant was then centrifuged 2,000×g for 30 min at 4° C. to remove debris. The media was mixed with an equal volume of 16% Polyethylene Glycol (PEG) and incubated overnight at 4° C. to enrich for exosomes within the EVs. Samples were centrifuged at 3000×g for 1 hour at 4° C. The supernatant was removed, and the tubes were decanted to remove excess PEG. The resulting EV pellet was suspended in 50-500 μL of PBS depending on the amount of starting media (20 μl PBS per ml of starting media) and stored at −80° C. For some applications, EVs were further purified by resuspending in PBS and centrifuging at 100,000 g for 1 hour at 4° C.

### BCA Protein Assay

Protein content was measured with the Micro BCA™ Protein Assay Kit (Thermo Fisher) using bovine serum albumin (BSA) standards. First, 50 μl of EVs diluted 1:10 in PBS was added to each well of 96 well plate. Then, 200 μL of working reagent was added to each well and mixed on a plate shaker for 30 seconds. The samples were incubated at 37° C. for 30 minutes and the absorbance was measured at 562 nm on BioTek a plate reader.

### Nanoparticle Tracking Analysis

Nanoparticle tracking analysis (NTA) was performed on EVs samples in triplicate using a Nanosight NS300 (Malvern Panalytical, Malvern, United Kingdom) equipped with 642 nm laser. Three videos of 60 sec each were taken for each measurement. Analysis of the resulting videos was done using the NTA 3.4 software provided by Malvern Panalytical.

### Transmission Electron Microscopy (TEM)

Intact EVs were prepared for negative stain TEM using the drop-by-drop method. Freshly glow discharged 300 mesh carbon coated Cu grids (Electron Microscopy Sciences) were floated onto 5 μL of EV drops for 20 seconds. Grids were then washed twice by briefly touching the grid to the top of 50 μL drops of ddH₂O. Excess solution was wicked off the grid gently using the edge torn filer paper between each wash, while avoiding direct contact with the coating side. Grids were negative stained on a 5 μL drop of 2% uranyl acetate (Electron Microscopy Sciences) for 5 sec followed by blotting on filter paper. The samples were imaged on a Tecnai T12 transmission electron microscope operating at 100 kV using an AMT CMOS camera.

### Proteomic Analysis of EVs

After two additional washing steps, the iNP-EVs were resuspended in lysis buffer and then sonicated on ice. The lysate was centrifuged at 4,500×g for 10 min and the resulting supernatant was centrifuged at 10,000×g for 1 h at 4° C. The pellet was then resuspended in RIPA buffer. The membrane fraction was solubilized by rotating the sample on a rotisserie sample mixer for 1 h at 4° C. The solubilized membrane fraction was then collected after centrifugation at 21,000×g for 10 min at 4° C. The membrane fraction was then subjected to proteomic analysis.

### AKI Models Using Immortalized Human Tubule Cells

Immortalized human cortical and proximal tubular epithelial (HK-2) cells were cultured in DMEM-F-12 with FBS [10% (wt/vol)] (Life Technologies, Carlsbad, CA), Insulin-Transferrin-Selenium (ITS) (Corning, Corning, NY), hydrocortisone (0.1 μM), and 1% Penicillin/Streptomycin. To induce nephrotoxic AKI, cells were treated with a 0.693 mg/mL solution of cisplatin (MilliporeSigma) dissolved in 0.9% NaCl to a final concentration of 10 μM for 48 h. To induce the sepsis-associated AKI model, cells at confluence were exposed to 1 mg/ml purified cell-free hemoglobin (CFH) for 48 h. For all experiments, we used CFH purified from normal human blood by high pressure liquid chromatography (Pishchany, G., et al. J Vis Exp. 2013 Feb. 7 (72): 50072). After 48 h, the control or treated cells were harvested for immunostaining or assays. In both models, culture media was replaced after 48 h. For iNP-EV treatment, 10 μg of EVs was added to cells for the next 24 h.

### Nephrotoxic AKI Model Using Human Induced Pluripotent Stem Cell Derived Kidney Organoids

Kidney organoids were generated from iPSCs (ATCC, DYR0100 (origin: SCRC-1041 foreskin fibroblast cell line)) using an established protocol (Takasato, M., et al. Nature 2015 526:564-568; Przepiorski, A., et al. 2018 Stem cell reports 11:470-484). Briefly, iPSCs were grown on Geltrex-coated (Thermo Fisher Scientific, Waltham, MA) plates in mTesR medium (STEMCELL Technologies, Cambridge, MA) media. After reaching confluency, the iPSCs were dissociated with Accutase (Thermo Fisher Scientific), seeded onto Geltrex-coated plates at a density of 100,000 cells/cm² and treated with mTesR media with 10 μM ROCKi (STEMCELL Technologies) for the first 24 h. iPSCs were treated with mTesR media without ROCKi for the next 48 h. From day 3, iPSCs were treated with 8 μM Wnt activator CHIR (Reagents Direct, Encinitas, CA) for the first five days. The intermediate mesoderm cells derived were then treated with 200 ng/ml FGF9 (Peprotech, Cranbury, NJ) and 1 mg/ml Heparin (Sigma-Aldrich, St. Louis, Missouri) for two more days to differentiate to nephron progenitors. On day 7, the cells were dissociated and grown on transwell with a one-hour pulse of 5 μM Wnt activator CHIR followed with FGF9 treatment until day 12. The differentiated organoids were harvested between day 21-day 25 for further experiments or analysis.

Kidney organoids were treated with 5 μM CHIR for 48 h to induce toxicity. Culture media was replaced after 48 h. For iNP-EV treatment, 10 μg of EVs was added to cells for the next 24 h. Organoids were treated with iNP-EVs alone without cisplatin were also used for comparison.

### Animal Model of AKI

Animal studies were conducted following the guidelines for the Use of Laboratory Animals by the American Association for Laboratory Animal Science. All protocols were approved by the Institutional Animal Care and Use Committee (IACUC) (Protocol number: M1700172-01). Unilateral renal ischemia-reperfusion (I/R) with simultaneous contralateral nephrectomy surgery was conducted on 12-13 week old male FVB/NHsd mice (n=15) (Au-Skrypnyk, N. I., et al. J Vis Exp. 2013 Aug. 9; (78): 5049531). Anesthesia was induced and maintained by controlled inhalation of isoflurane in O₂. The mice were placed on a circulating water heating pad for body temperature maintenance and provided with lubricating eye ointment for corneal protection during the procedure. First, the right-sided nephrectomy was performed. An incision was made along the dorsal midline (approximately 1.5 cm) using scissors and forceps, followed by a small incision through the dorsal right flank muscle and fascia above the kidney and exteriorizing of the right kidney. Dissection of the upper and lower poles of the kidney freed them from surrounding tissue while preserving the adrenal gland. Then a 4-0 silk suture was tied around the hilum of the right kidney using a double surgical knot. The kidney is removed by cutting distal to the knot. The muscle layer was closed with 5-0 vicryl. Following this, a small incision was made through the left flank muscle and fascia, and the left kidney was exteriorized. While holding the kidney using blunt forceps, the renal pedicle was freed from surrounding fat. The left renal pedicle was clamped with a non-traumatic vascular clamp using holding forceps. The time of ischemic time was 22 minutes. The left kidney and incision were covered with sterile saline-soaked gauze and a section of metalized polyethylene terephthalate thermal blanket during the ischemic time. After the ischemic time was complete, the clamp was released. The kidney was pushed back into the retroperitoneal space. Vicryl 5-0 absorbable sutures closed the muscle layer, and nylon 4-0 nonabsorbable sutures closed the skin layer. Ketofen in sterile saline was provided via intraperitoneal injection for pain control for >48 h. The sham animals were administered flank incision with exposure of kidneys.

iNP-EVs Injection

A total of 16 animals were used for the study. Urine was collected from all the mice 1 week prior to the surgery. Before surgery, the animals were divided into three groups (I/R, I/R+EVs, Sham). The I/R was conducted and blood and urine were collected 24 h after the surgery. The mice in the I/R+EVs group were injected with 100 μg of iNP-EVs dissolved in PBS to final volume of 200 μl via the tail vein at 24 h after AKI surgery, while control I/R mice received PBS alone. The blood and urine samples were collected once per week for 2 weeks after surgery. Transcutaneous measurement of glomerular filtration rate (tGFR) was conducted using FITC-sinistrin 24 h and 2 weeks after the surgery. The mice were sacrificed 2 weeks after surgery and tissues (kidney, liver and spleen) were collected. The center of the kidney was kept at 4% PFA for paraffin sectioning whereas the poles were immediately placed in liquid nitrogen for tissue protein extraction. Paraffin sections of tissues were stained for periodic acid-schiff (PAS), Sirius Red, F4/80, and H&E. Slide scoring was performed by investigators blinded to groups.

### Immunocytochemistry

Organoids were fixed using 4% paraformaldehyde (Thermo Fisher Scientific) for 30 min. The organoids were then washed with PBS (Fisher Scientific). The organoids were then blocked with 10% donkey serum (Millipore Sigma) in PBS containing 0.03% Triton (Sigma-Aldrich) for 30 min. The organoids were washed with PBS and stained with primary antibodies (Table 1) overnight at 4° C. The next day, the organoids were washed with PBS and stained with corresponding secondary antibodies (Table 1). Hoechst/DAPI was used for nuclear staining. Fluorescently stained organoids were then imaged using the confocal microscope (Zeiss LSM710/Nikon Spinning Disk). Fluorescent images were analyzed using ImageJ software.

### Biochemical Assays

### MTT

The organoids were washed with PBS and then treated with MTT (3-(4,5-dimethylthiazol-2-yl)-2,5-diphenyltetrazolium bromide) reagent (Sigma-Aldrich) (1 mg/ml) for at least 60 min, leading to the purple formazan formation in living cells. The reagents were then removed and isopropanol was added to dissolve the insoluble formazan. The absorbance of the colored solution was measured using a microplate reader at 560 nm and 690 nm. Background absorbance at 690 nm was subtracted from absorbance at 560 nm and the O.D. values were plotted to measure cell viability.

### LDH (Lactate Dehydrogenase)

LDH release from the cells was evaluated using CyQUANT™ LDH Cytotoxicity Assay Kit. Briefly, cell culture medium was collected for other purposes at 48 h after the treatment. The cells were treated with 10% Triton X-100 and incubated for 15 min before to obtain the maximum LDH release from the sample. Cell culture media was collected and 50 μl of each sample was transferred into a 96-well opaque-walled, non-transparent assay plate. The LDH detection reagent was prepared by combining the LDH detection enzyme mix and reductase substrate. Then 50 μl of detection reagent was added to each well. The wells were incubated for 60 min at room temperature. Luminescence was recorded on a plate reader at 490 nm and the corresponding O.D. was plotted to quantify cell death.

### CASPASE 3/7 Activity

Caspase 3/7 activity in HK-2 cells was assayed using the Image-iT™ LIVE Red Caspase Detection Kit (Thermo Fisher Scientific). First, 1×FLICA (Fluorochrome Inhibitor of Caspases) Reagent solution was added to cells. Cells were then covered and incubated at 37° C. with 5% CO₂ for 1 h. The solution was then removed, and cells were gently rinsed with cell culture media. Cells were washed twice with 2 mL of 1× kit wash buffer, counterstained with Hoechst and imaged immediately.

### ELISA

The concentration of KIM-1 in the cell culture supernatant was determined using a Human Serum TIM-1/KIM-1/HAVCR Quantikine ELISA Kit (R&D Systems, Minneapolis, MN) following the manufacturer's protocol. Briefly, the cell culture media was diluted using the calibrator diluent in the kit (1:4) and then added to microplates pre-coated with human KIM-1. Treatment with series of solutions/washes resulted in a yellow product. Absorbance was measured using a plate reader at 495 nm and compared to the Quantikine kit standards.

### Assessment of In Vivo Renal Function

Urea assay: Blood was collected and centrifuged at 1000×g for 5-10 min. The separated serum was stored at −80° C. The serum was diluted with deionized water (1:5) for the urea assay using QuantiChrom™ Urea Assay Kit following the protocol. Briefly, 5 μl of diluted serum samples and standard were transferred to a 96 well plate. Then 200 μl of working reagent was added to the wells and samples were incubated 20 min at room temperature. The optical density was measured using a plate reader at 520 nm. The urea concentration was calculated using the provided formula.

Serum creatinine assay: Blood was collected via submandibular puncture with a 5 mm lancet in Microvetter CB 300 Z serum collection tubes (Braintree Scientific Inc., Braintree, MA) and allowed to coagulate for 30 min at room temperature. The clot was removed via centrifugation at 1000×g for 5-10 minutes. The resultant supernatant was removed and stored at −80 C for future analysis. Serum aliquots of 10 μl from each sample was packaged and shipped on dry ice to the O'Brien Kidney Center at the University of Alabama-Birmingham for serum creatinine measurements.

Transcutaneous measurement of glomerular filtration rate (tGFR): tGFR measurements were conducted following the established method (Scarfe, L., et al. J Vis Exp. 2018 Oct. 21 (140): 58520). Briefly, 40 mg/mL FITC-sinistrin was prepared in phosphate buffered saline (PBS) without calcium and magnesium for injection. After weighing each mouse, the amount of FITC-sinistrin required to administer 0.15 mg FITC-sinistrin per gram body weight was calculated. tGFR devices were attached to mice under anesthesia. The body temperature of each animal was continuously maintained using a heating mat. To prevent autofluorescence of the mouse hair, Nair depilation cream was used to remove all hair from the mice right top from the hind legs to neck area followed by wiping with water to completely remove the cream and hair. The cleaned area was then wiped with Prevantics chlorhexidine gluconate (3.15%) and isopropyl alcohol (70%) swabs (Professional Disposables International, Inc. Orangeburg, NY). Adhesive tape was attached to the tGFR devices which were then connected to batteries. Using silk tape, the sensors were securely attached to the mice and the baseline measurement was taken for 3 min as the tail was dipped in warm water. After 3 min, the FITC-sinistrin was injected into the mice via tail vein injection. The mice were then moved to a new cage to recover from isoflurane anesthesia where the measurements were taken for 90 more min. After 90 minutes, the sensors were detached from the mice and the measurements were collected using then Mediabeacon software. The FITC sinistrin half-life was obtained and used to calculate the tGFR for each animal.

Urinary NGAL, KIM-1: The urine collected was centrifuged at 6000×g for 5 min and kept at −80° C. The supernatant of the urine was transferred to another tube. The concentration of NGAL and KIM-1 in the urine was determined using a commercially available Mouse Lipocalin-2/NGAL and KIM-1 Quantikine ELISA Kit (R& D Systems,) following the protocol. Briefly, the urine was diluted using the calibrator diluent in the kit (1:20). The assay uses the quantitative sandwich enzyme immunoassay technique to determine the NGAL concentration. Standards, control, and samples were pipetted into the wells of a microplate coated with antibody specific for mouse Lipocalin-2. Then an enzyme linked to the mouse Lipocalin-2 antibody was added to the wells. Lastly, a substrate solution was added which results in an enzyme reaction leading to a blue product. The acidic stop solution was added to stop the reaction and yellow product intensity was measured using a plate reader at 495 nm.

Human inflammatory cytokine assay: The amount of pro-inflammatory cytokines (IL-1β, IL-6, IL-8, IFN-γ) present in cell supernatants from iNP-EV-treated or untreated organoids was determined using a human inflammatory cytokine kit (BD™ Cytometric Bead Array (CBA)), following the instructions of the manufacturer. A FACS flow cytometer (BD) was used to analyze the samples.

### Histology

H&E staining: To assess histological injury and fibrosis hematoxylin and eosin (H&E) staining was performed. Deparaffinized and hydrated slides were treated with xylene following with ethanol wash. Hematoxylin & eosin was added to the slides respectively with rinsing in between. The slides were then clarified and blued, then dehydrated. After final wash the slide were observed through microscope. The slides were coversliped in a resinous medium.

Picrosirius red staining: First, a solution of 0.1 g Sirius red F3BA per 100 ml picric acid was made. Deparaffinized and hydrated slides were then incubated with the picrosirius red solution for 45 minutes. The slides were rinsed and washed with alcohol and xylene.

PAS staining: Deparaffinized and hydrated slides were placed in 0.5% periodic acid and rinsed with water. Schiff's reagent was then added and washed and then placed in Richard-Allan Hematoxylin 7211. The slides were then clarified and blued, then dehydrated and covered.

hiPSC-Derived Podocytes Exposed to High Glucose and Treated with INP-EVs

Podocytes were derived from hiPSCs using our published protocol and treated with high concentration (100 mM) of glucose for 48 hours (Bejoy, J., et al. Stem Cell Research & Therapy 2022 13:355). Glucose-treated podocytes were subsequently given 5 μg of iNP-EVs for 24 hours after glucose treatment. The impact of EVs on podocyte apoptosis was evaluated using flow cytometry analysis for Annexin V and cell permeability.

TABLE 1
Ordering information for key reagents.

| Reagent | Company | Catalog # |
|---|---|---|
| DMEM/F-12, HEPES | Gibco | 11330-032 |
| ITS | Sigma | I1884 |
| ITS | Fisher Scientific | 41400-045 |
| PenStrep | Gibco | 15070-063 |
| Hydrocortisone | Sigma | H0888-1G |
| GlutaMax | Gibco | 35050-061 |
| DMEM/F12 for NPSR | Invitrogen | 11330-032 |
| NEAA | Fisher Scientific | |
| 2-Mercaptoethanol | Fisher Scientific | AC125470100 |
| PenStrep | Corning | 30002CI |
| B-27 Supplement | Invitrogen | 12587-010 |
| PEG | Sigma-Aldrich | 81260 |
| FBS | Fisher Scientific | 10-437-028 |
| PBS | Mediatech/CellGro-Corning | 21-040 CM |
| Vitamin A | Fisher Scientific | 12-587-010 |
| BMP7 | Fisher Scientific | 354-BP-010 |
| FGF2 | Peprotech | 100-18B |
| Heparin | Sigma | H3149-100KU |
| Y27632 | Stemcell Technologies | 72304 |
| Human LIF | Millipore | LIF1050 |
| CHIR99021 | Reagents Direct | 27-H76 |
| LDN193189 | Reagents Direct | 36-F52 |
| A83-01 | APExBIO | A3133 |
| DAPI | Sigma-Aldrich | D9542 |
| Shaker | Thermofisher Scientific | MaxQ 2000 |
| Zoe ™ Fluorescent Cell | Bio-Rad | 1450031 |
| Imager | | |

TABLE 2
Antibodies and dilutions

| | Primary | Origin/ | Supplier/ | |
|---|---|---|---|---|
| Cell type | Antibody | Isotype | Catalogue # | Dilution |
| EV | CD63 | Rabbit IgG | Santa Cruz | 1:100 |
| markers | | | Biotechnology/sc- | |
| | | | 5275 | |
| | CD81 | Rabbit IgG | Santa Cruz | 1:200 |
| | | | Biotechnology/sc- | |
| | | | 166028 | |
| | ALIX | Rabbit IgG | Santa Cruz | 1:100 |
| | | | Biotechnology | |
| | TSG101 | Rabbit IgG | Santa Cruz | |
| | | | Biotechnology | |
| Reporter | mCherry | Rabbit IgG | Cell Signaling | 1:200 |
| | | | Technology/43590S | |
| | RFP | Rabbit IgG | MBL International/ | 1:200 |
| | | | PM005 | |
| Nephron | CITED1 | Mouse IgG2a | Fisher Scientific/89- | 1:100 |
| Progenitors | | | 335-107 | |
| | SIX2 | Rabbit IgG | Proteintech/11562- | 1:200 |
| | | | 1-AP | |
| | PAX2 | Goat IgG | R and D | 1:100 |
| | | | systems/AF3364-SP | |
| Proliferation | Ki-67 | Rabbit IgG | Abcam/ab16667 | 1:200 |
| marker | | | | |
| Apoptosis | Cleaved | Rabbit IgG | Cell signaling | 1:200 |
| marker | caspase 3 | | Technology/9664 | |
| | (CC-3) | | | |
| Kidney | KIM-1 | Goat IgG | R and D systems/ | 1:200 |
| injury | (human) | | AF1750-SP | |
| marker | KIM-1 | Goat IgG | R and D systems/ | 1:200 |
| | (mouse) | | AF1817 | |
| Actin | F-Actin | Molecular | Thermo | 1:200 |
| Cytoskeleton | | Probes Alexa | Scientific/A12381 | |
| | | Fluor 594 | | |
| | | Phalloidin | | |
| Nephron | E- | Mouse IgG1 | Fisher | 1:200 |
| segment | Cadherin | | Scientific/ | |
| | | | BDB610181 | |
| markers | GATA3 | Goat IgG | R and D systems/ | 1:200 |
| | | | AF2605-SP | |
| | LTL | Flourescein | Vector | 1:200 |
| | | Lotus | Laboratories/FL- | |
| | | Lectin | 1321 | |
| | SLC3A1 | Rabbit IgG | Proteintech/16343- | 1:200 |
| | | | 1-AP | |
| | PODXL | Goat IgG | R and D | 1:200 |
| | | | systems/AF1658 | |

| | | | |
|---|---|---|---|
| Reporter | Alexa 488, donkey | Life | 1:400 |
| | anti-goat IgG | Technologies/A- | |
| | | 11055 | |
| | Alexa 594, goat | Life | 1:400 |
| | anti-rabbit IgG | Technologies/A- | |
| | | 11037 | |
| | Alexa 647, donkey | Life | 1:400 |
| | anti-goat IgG | Technologies/A- | |
| | | 32849 | |

### Results

Generation of Induced Nephron Progenitor Cells (INPs) from Reprogrammed Human Kidney Epithelial Cells (HK2-C8).

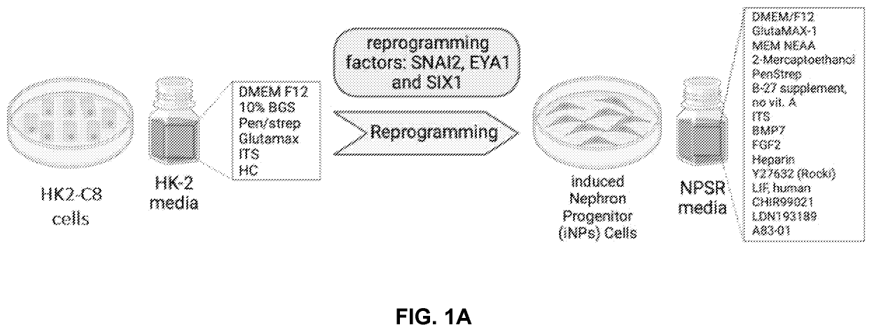

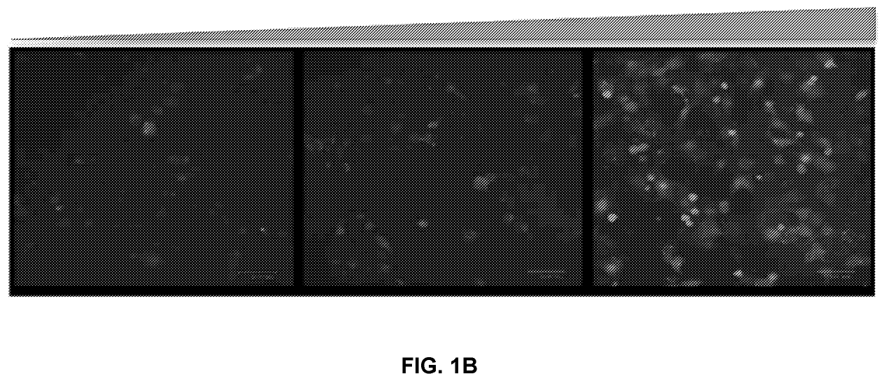

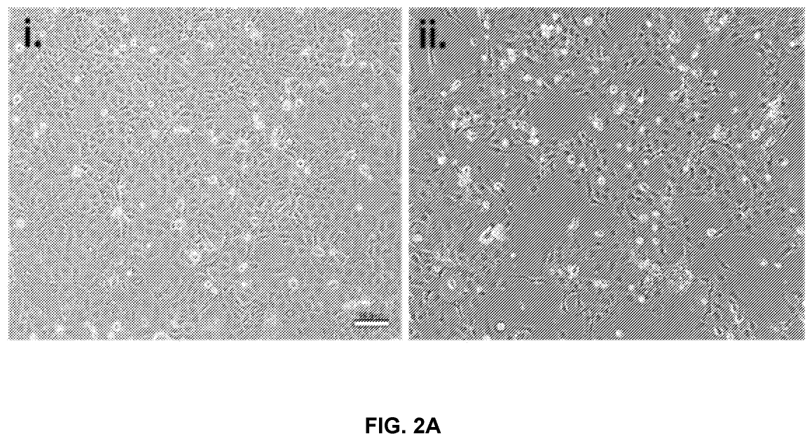

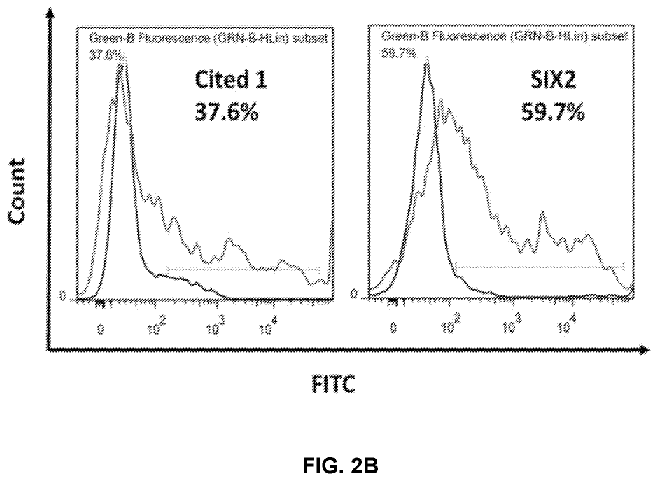

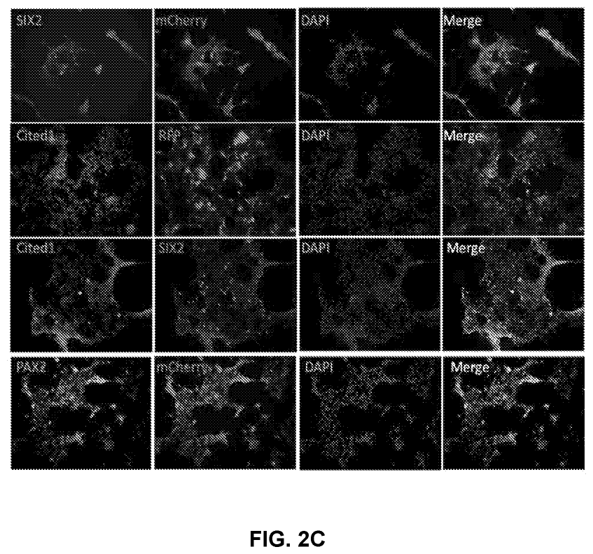

HK2-C8 cells were exposed to doxycycline to induce reprogramming process, followed by NPSR media to support the iNP phenotype (FIG. 1A). The reprogrammed HK2-C8 cells started to become mCherry+ in a few cells beginning one day after reprogramming with doxycycline. The cells changed morphology during reprogramming (FIG. 2A) and the mCherry expression levels increased with doxycycline exposure until the cells were harvested (FIG. 1B). HK2-C8 treated with NPSR containing doxycycline-maintained mCherry expression, suggesting the expression of the reprogramming transcription factors SNAI2, EYA1, and SIX1. The morphology of the cells changed to more elongated structures after treatment with the NPSR was observed (FIG. 2A). The cells were harvested at day 8 and co-stained for mCherry/RFP from the transposon with the NP markers PAX2, SIX2 or CITED1 (FIG. 2C). Co-expression of the NP markers with the mCherry reporter was found in the reprogrammed cells at Day 8 (FIG. 2C). Flow cytometry analysis revealed that iNPs expressed around 40% and 60% of CITED1 and SIX2, respectively (FIG. 2B).

Isolation and Characterization of iNP-EVs.

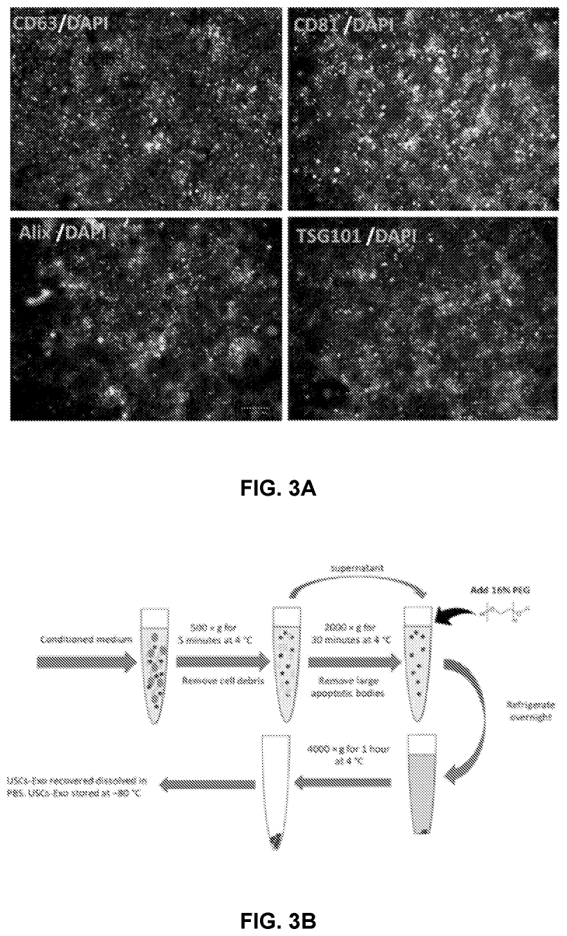

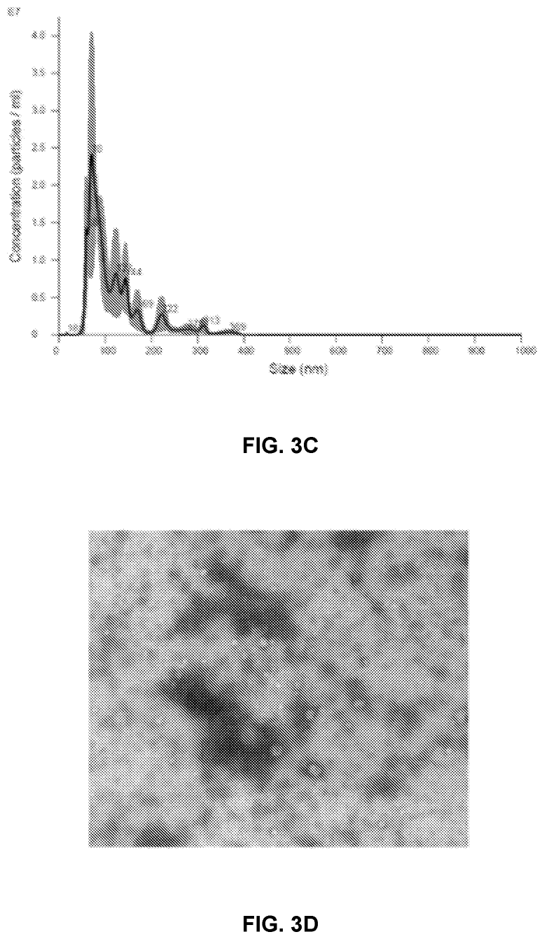

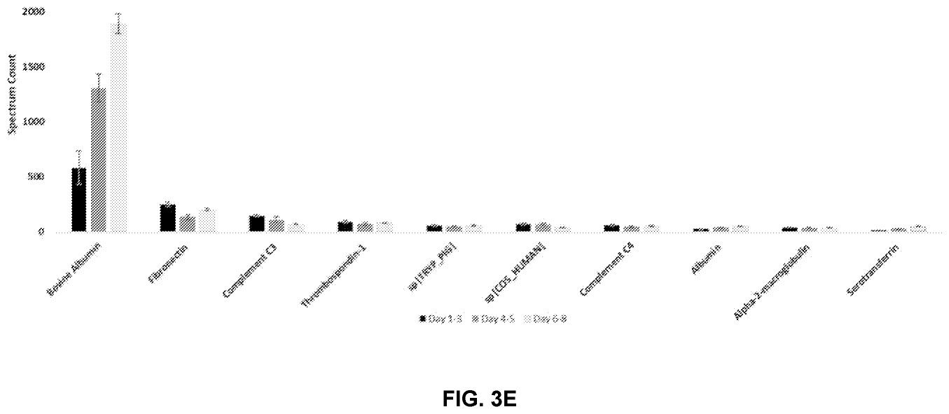

Immunostaining with exosome markers such as CD63, CD81, Alix, and TSG 101 showed prominent expression of these markers in the iNPs (FIG. 3A). Conditioned NPSR media was collected from the iNPs grown on 10 cm dishes from day 5 to day 8 of the reprogramming process. The EVs were isolated using the ExtraPEG method (FIG. 3A) and resulting EVs were characterized using NTA analysis (FIG. 3B). EVs isolated from iNPs had an average size of 116.2 nm+/−6.6 nm with the mode size of the particles being 72.7 nm+/−9.2 nm (FIG. 3C). The EVs had a concentration of 1.30×10⁹ particles/ml+/−2.39×10⁸ particles/ml and the protein content ranged from 18-20 μg/ml of media (FIG. 3C). Electron microscopy was used to confirm the presence of EVs in the samples (FIG. 3D). Images showed small spherical particles indicating that EVs had been retrieved and confirming the NTA analysis (FIG. 3D). Proteomic analysis of the iNP-EVs showed increased expression of many proteins including fibronectin, complement factors, macroglobulin, and serotransferrin (FIG. 3E).

INP-EVs Decreased Apoptosis and Increased Proliferation of Human Tubule Cells Treated with Cisplatin

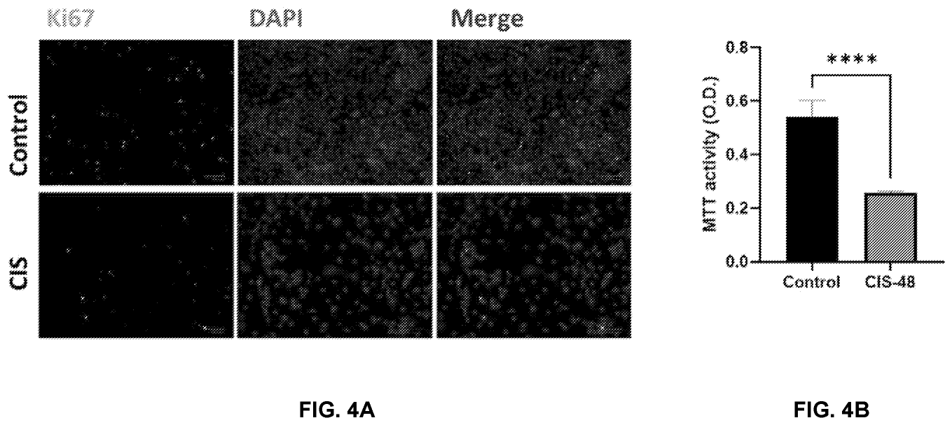

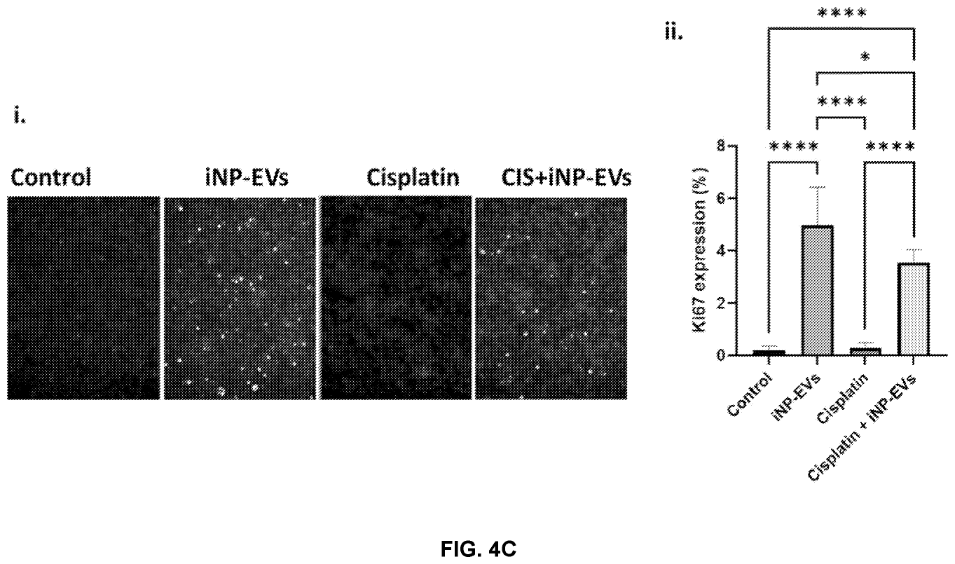

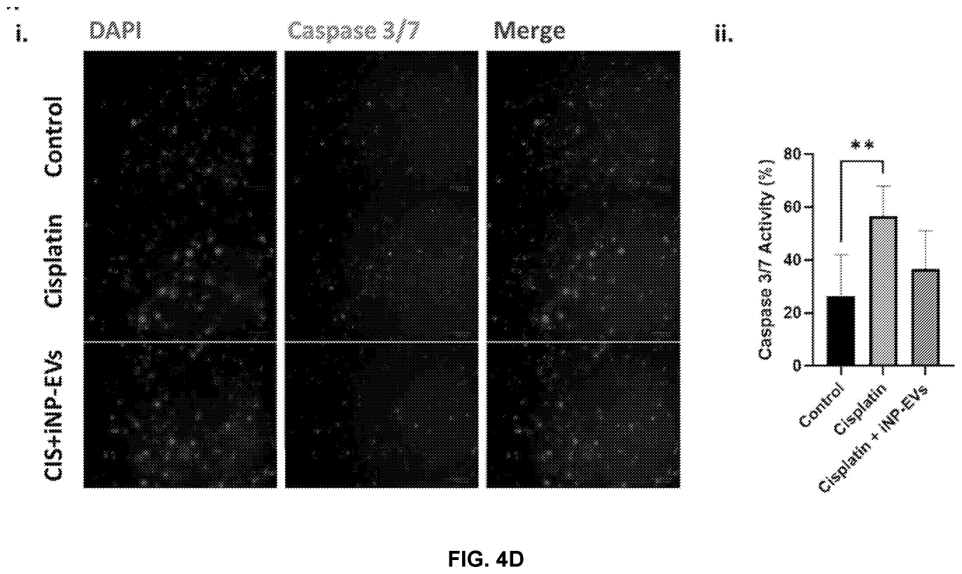

First, cisplatin-induced nephrotoxicity was confirmed in immortalized human tubule cells (HK-2). The cisplatin treatment reduced the Ki-67+ proliferating cell population (FIG. 4A) as well as viability in the cells (FIG. 4B). Cisplatin also caused an increase in Caspase 3/7 activity (FIG. 4Dii). To determine whether iNP-EVs promoted proliferation, HK-2 cells were injured and treated with or without iNP-EVs, then assayed for Ki-67 (green), a common biomarker for cellular proliferation (FIG. 4Ci). Cells were counterstained with Hoechst to stain the nuclei (blue). Following image quantification, we found higher numbers of Ki-67+ proliferating cells with the iNP-EVs treatment in comparison to the cisplatin-only group, suggesting that iNP-EVs enhanced cellular proliferation in the cultured immortalized tubule cells (FIG. 4Cii). In addition, iNP-EV treatment caused a decrease in Caspase 3/7 activity, suggesting that the iNP-EVs lowered apoptosis in tubule cells (FIG. 4Di, ii).

INP-EVs Improved Viability of Renal Tubular Epithelial Cells Treated with Cell-Free Hemoglobin

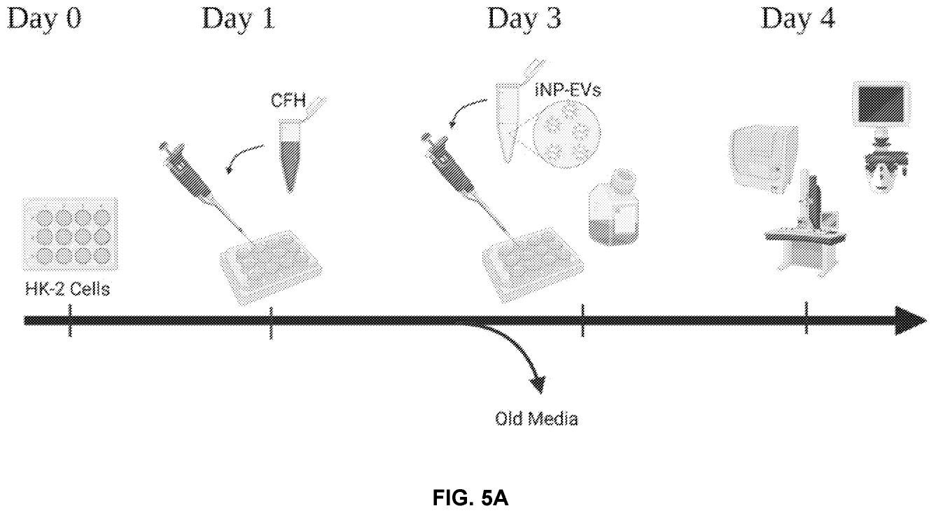

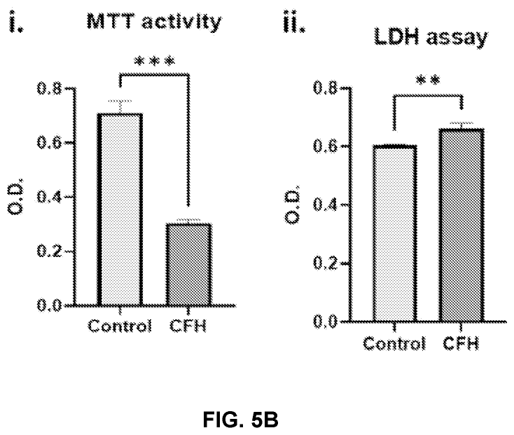

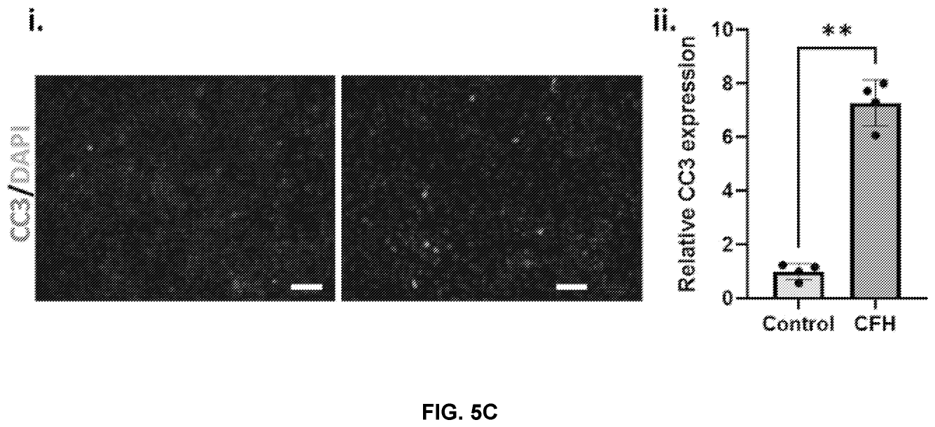

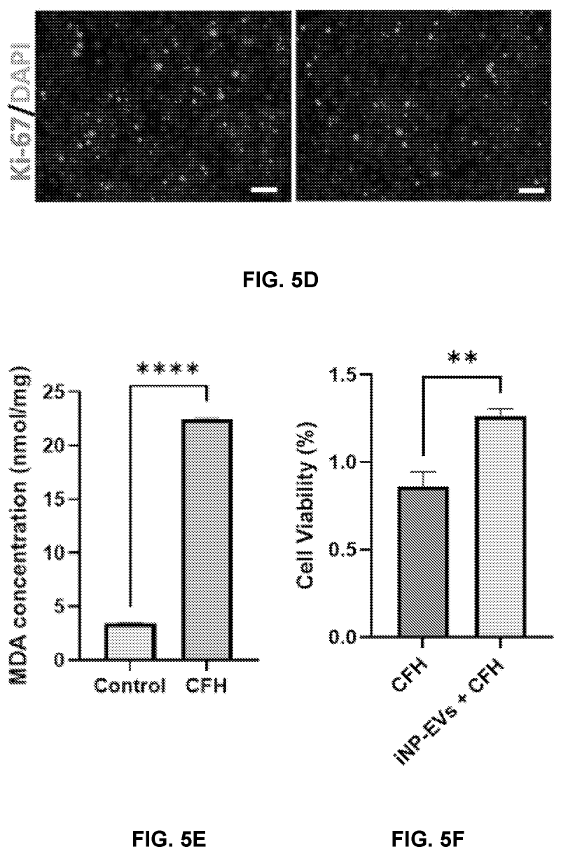

CFH increased cytotoxicity in renal tubular cells (HK-2) (Shaver, C. M., et al. Am J Physiol Renal Physiol 2019 317: F922-f929). Viability was examined by 3-(4,5-dimethylthiazol-2-yl)-2,5-diphenyltetrazolium bromide (MTT) assay and the CFH-treated group had a substantial decline in viability (p=0.0001; FIG. 5Bi). There was a higher LDH release in the CFH-treated groups (p<0.005; FIG. 5Bii). Next, the CFH-treated cells were stained with apoptosis and proliferation markers to understand the mechanism of cytotoxicity. Proliferation as evidenced by Ki-67 staining was not different between the groups (FIG. 5D). However, cleaved caspase-3 staining was increased in the CFH-treated group (FIG. 5Ci,ii). These results suggest that CFH can induce cytotoxicity in tubule cells via induction of apoptosis. Since Heme redox cycling between ferric and ferryl states generates globin radicals that cause lipid peroxidation (Reeder, B. J. & Wilson, M. T. Curr Med Chem. 2005 12 (23): 2741-51), we measured levels of peroxidation in CFH-treated HK-2 cells. CFH treatment increased the concentration of Malondialdehyde (MDA) from ˜3.5 μM to 22.5 μM (p<0.0001; FIG. 5E). To determine whether iNP-EVs promoted viability of CFH injured HK-2 cells, groups treated with or without iNP-EVs were assayed for MTT. The result showed that iNP-EVs treatment significantly increased tubule cell viability compared to the CFH-alone group (FIG. 5F).

### INP-EVs Reduced Injury, Inflammatory Cytokine Release, and Improved Proliferation in Cisplatin-Treated Human Kidney Organoids

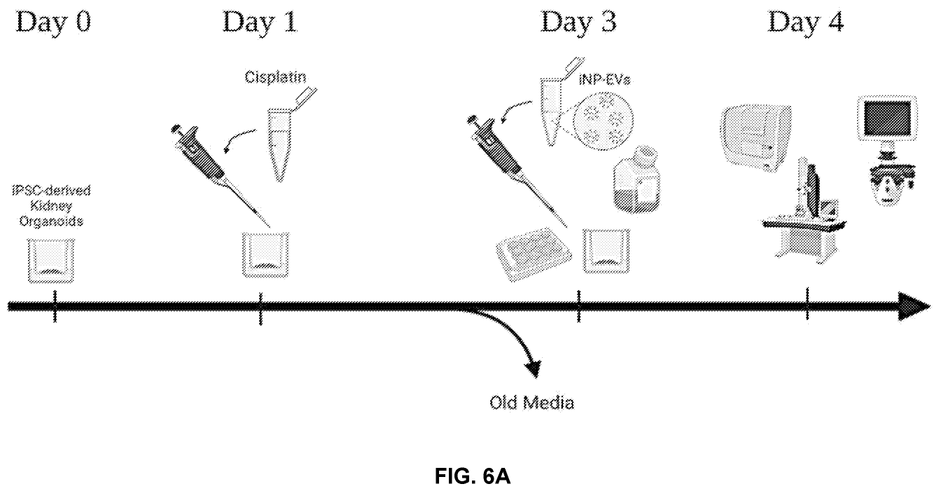

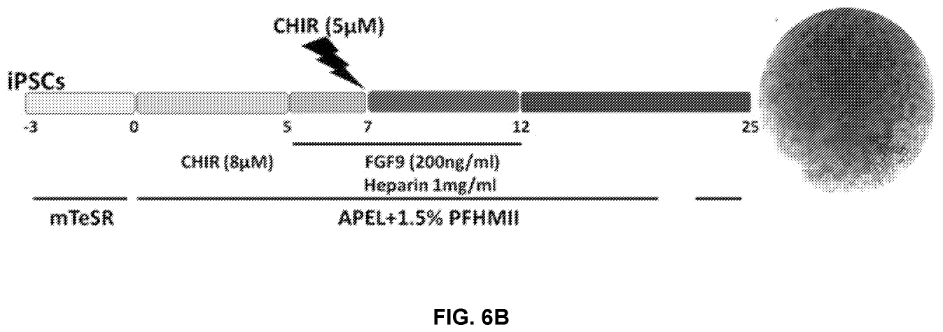

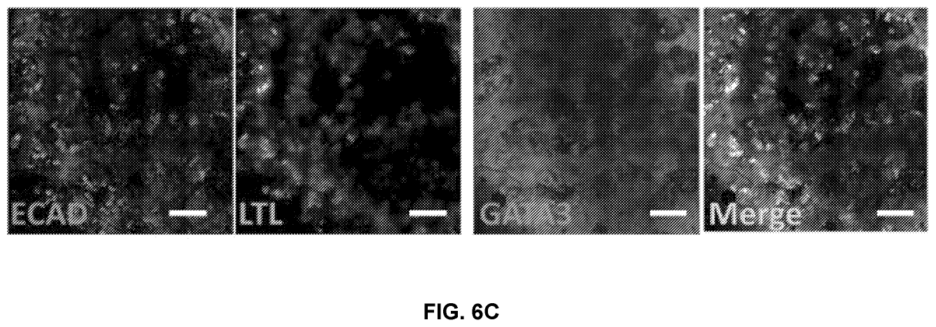

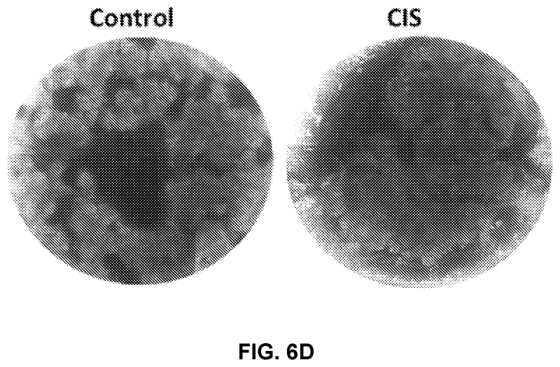

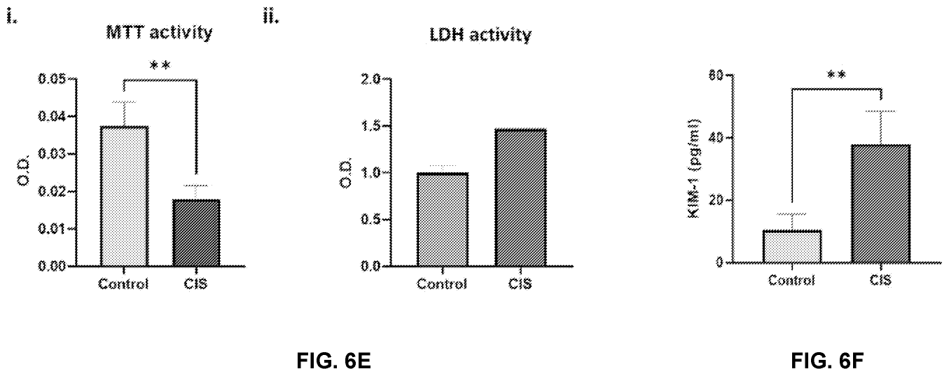

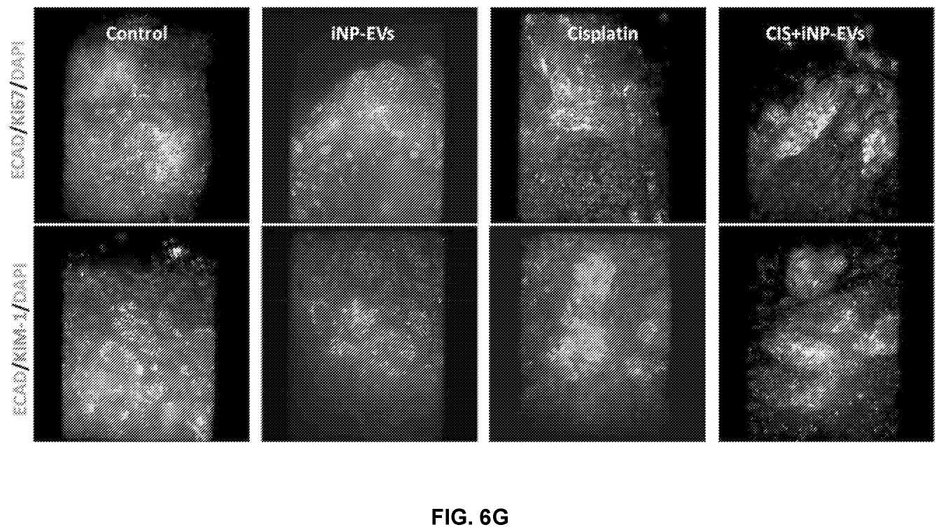

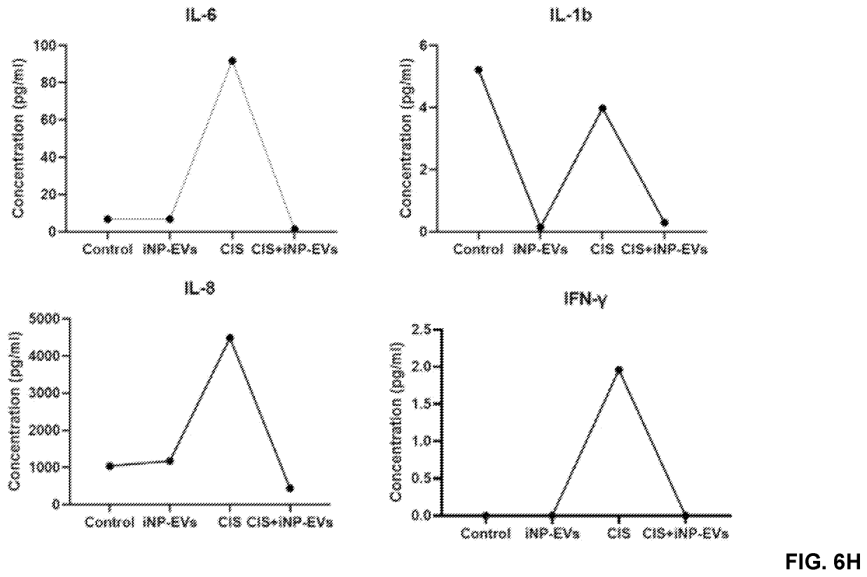

The Takasato transwell-based method was used to create human kidney organoids (FIG. 6B) (Takasato, M., et al. Nature 2015 526:564-568). The process involves generating intermediate mesoderm from primitive streak using the Wnt activator CHIR followed by induction of nephrogenesis with FGF9. During nephrogenesis, cells grown in a monolayer were harvested and transferred onto the transwells as a cell pellet to create an air-liquid interface for the rest of the culture time until day 25. Brightfield images of the day 25 organoids revealed s-shaped structures and renal vesicles (FIG. 6B). Immunostaining analysis of the mature organoids confirmed the presence of distal tubule cells (ECAD), proximal tubule cells (LTL), and collecting duct (GATA3; FIG. 6C) (Bejoy, J., et al. J Am Soc Nephrol. 2022 33 (3): 487-501). The efficiency of differentiation was 60-70% as determined by whole mount staining. After treatment with cisplatin for 48 hours, organoids were examined for toxicity and viability. The tubule segments were disrupted in the group receiving cisplatin, indicating injury (FIG. 6D). Viability was also significantly reduced after cisplatin treatment (p<0.01; FIG. 6Ei). The amount of cytotoxicity was higher in the cisplatin-treated group than the control group (FIG. 6Eii). Exposure to cisplatin increased the amount of kidney damage marker KIM-1 that was released into the cell culture media as measured by ELISA (p<0.01; FIG. 6F). To determine whether iNP-EVs promoted cellular proliferation in cisplatin-injured kidney organoids as they did in the HK-2 cells, the groups treated with or without iNP-EVs were stained for Ki-67. The result showed that iNP-EVs treatment increased the Ki-67+ population in the iNP-EVs group as compared to the cisplatin alone group (FIG. 6G). The change in kidney injury was also analyzed using kidney injury marker KIM-1. There was a reduction in KIM-1 in the iNP-EVs group as compared to cisplatin alone group (FIG. 6G). The cytokines produced in the media was next examined in four groups: no treatment, iNP-EVs alone, cisplatin alone, and cisplatin treated with iNP-EVs. Four different cytokines were expressed differentially in the cisplatin group as compared to the control group. These were IL6, IL1β, IL8 and IFNγ. The INP-EVs treatment reduced the expression of each these proinflammatory cytokines as compared to the cisplatin alone group (FIG. 6F).

Effect of iNP-EVs in the Injury Biomarkers within 48 Hours of Treatment in I/R Mice.

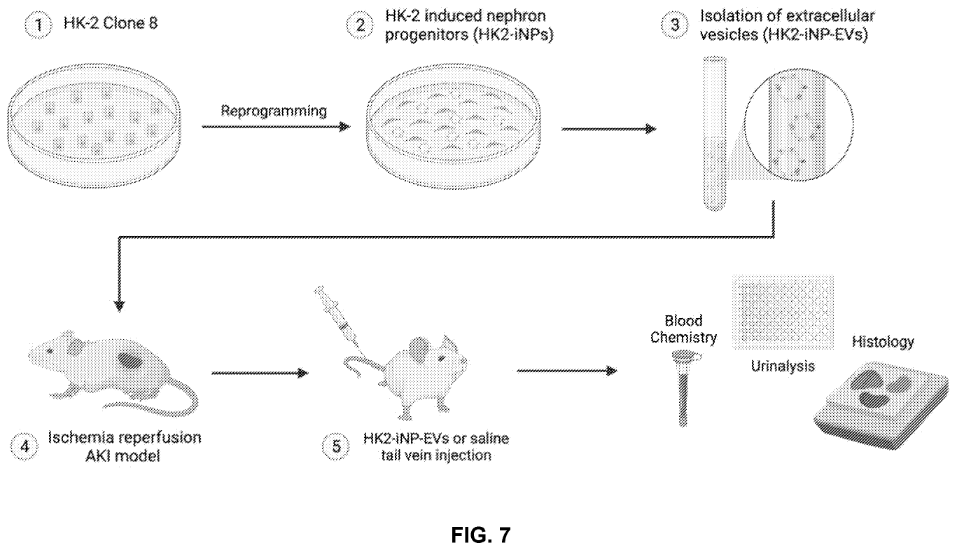

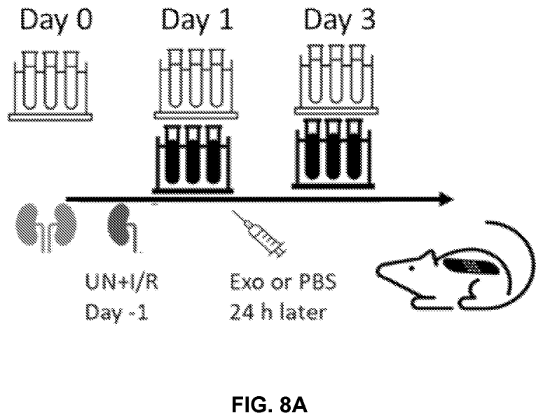

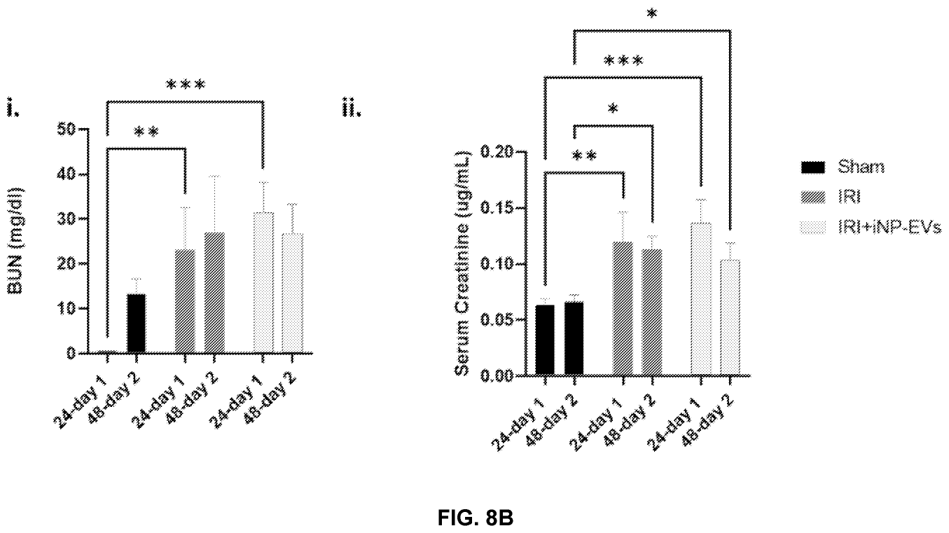

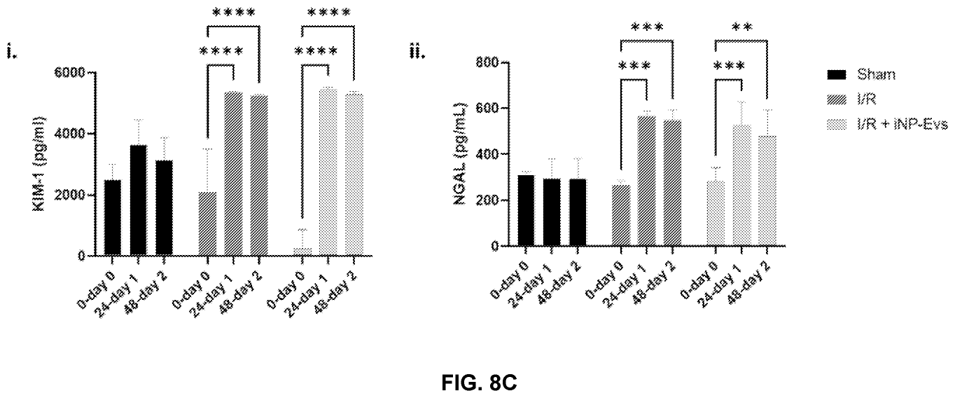

Effect of iNP-EVs in the mouse AKI models were conducted following established methods (FIG. 7). Urine and blood were collected at Day 1 before iNP-EVs treatment and at Day 2 after the iNP-EVs treatment (FIG. 8A). The levels of serum BUN and sCr were increased in both groups 24 hours after surgery, indicating successful injury (FIG. 8Bi,ii). To evaluate the effects of iNP-EVs on prevention of tubule injury in an I/R mouse model of AKI, ELISA to quantify levels of the kidney injury biomarkers KIM-1 and NGAL in the urine was conducted. It was determined by KIM-1 ELISA that iNP-EVs slightly reduced the injury marker expression compared to the I/R control group (FIG. 8i). Additionally, the NGAL ELISA also showed the same trend (FIG. 8ii).

INP-EVs Reduced the Injury Marker Expression as Well as Improved Proliferation in Tubule Cells within 48 Hours in I/R Mice.

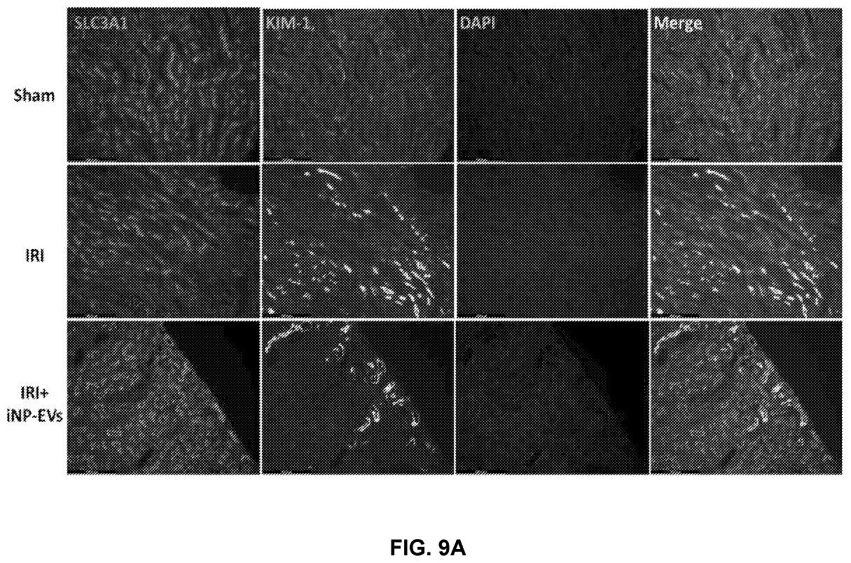

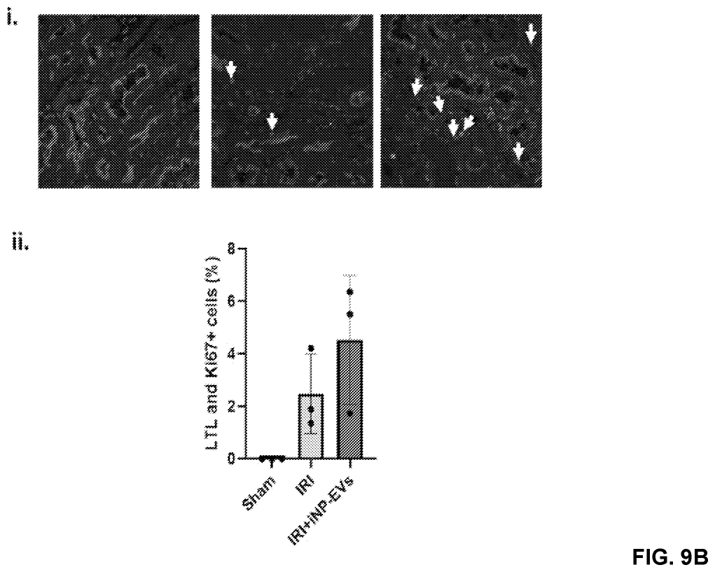

The change in kidney injury was analyzed using kidney injury marker KIM-1 and results indicated reduction in the I/R+ INP-EVs group compared to I/R group (FIG. 9A). To determine whether iNP-EVs promoted proliferation, sections were stained from mice that were treated with or without iNP-EVs for Ki-67. Quantification of the images showed elevated marker of proliferation Ki-67 staining that was colocalized with LTL+ tubules in mice receiving the iNP-EVs treatment, suggesting that iNP-EVs have a major effect on tubular proliferation.

Changes in the Tissue Morphology after Treatment with iNP-EVs for 48 Hours in I/R Mice.

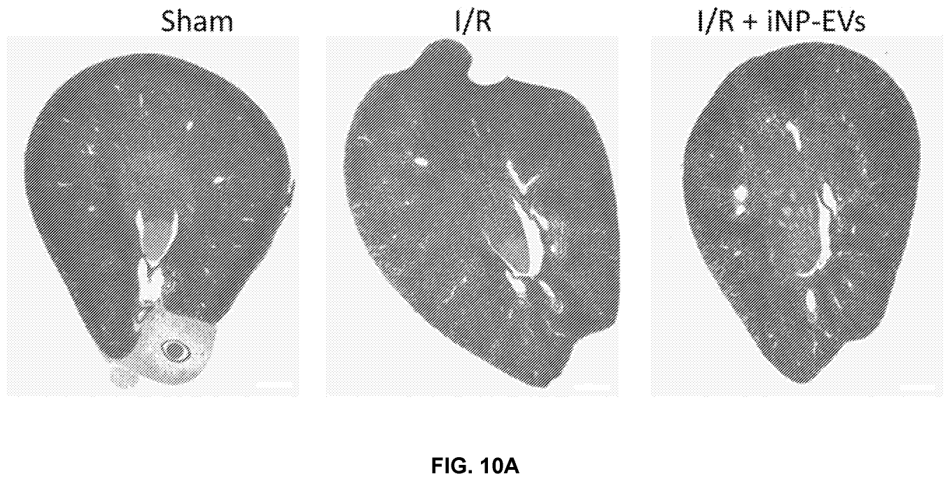

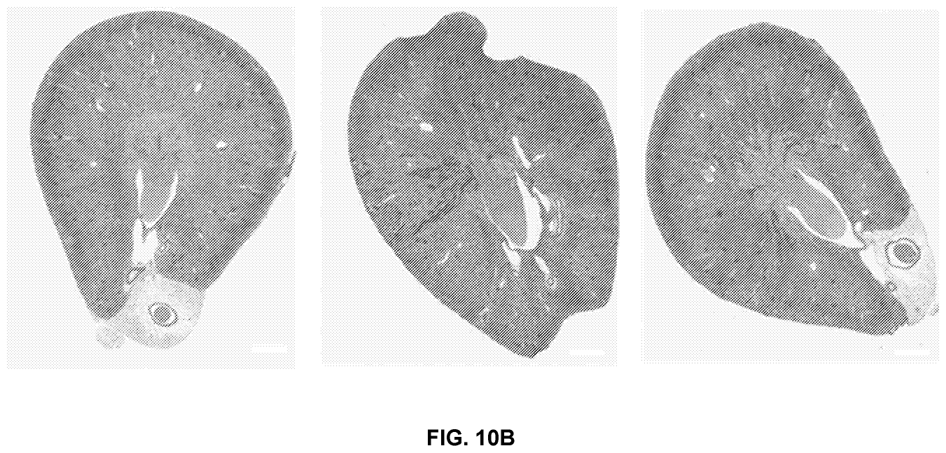

Tubular injury was defined as tubular vacuolization, loss of the brush border, tubular dilation, and/or tubular cast formation in the IHC images. There was visible decrease of casts in the I/R+iNP-EVs kidney tissue compared to the I/R alone group. I/R alone samples had more casts compared to the sham whereas I/R+iNP-EVs group were comparable to sham group (FIG. 10A). There was also reduction in PAS staining in I/R+iNP-EVs group compared to IRI group suggestive of less basement membrane damage (FIG. 10B).

### INP-EVs Reduced the Injury Biomarkers and Supported Survival of I/R Mice Over Two Weeks.

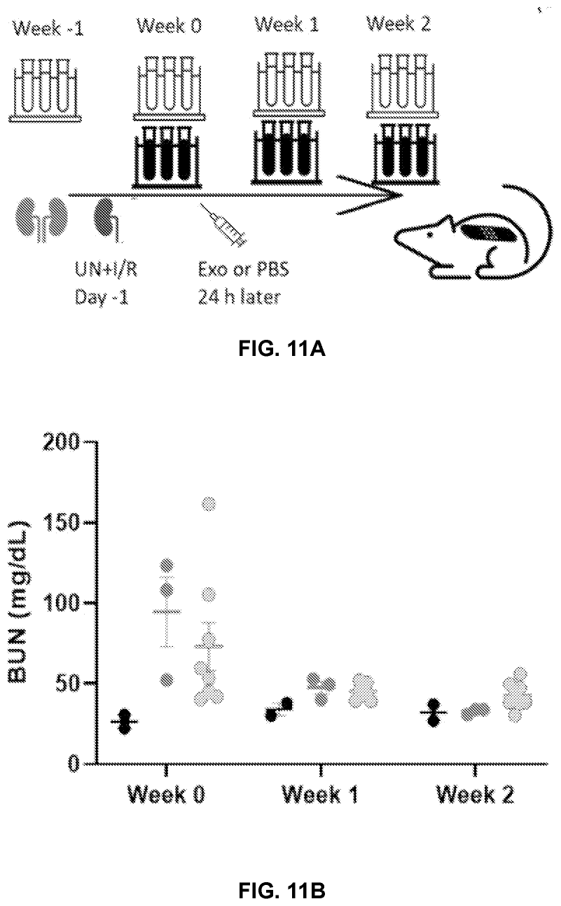

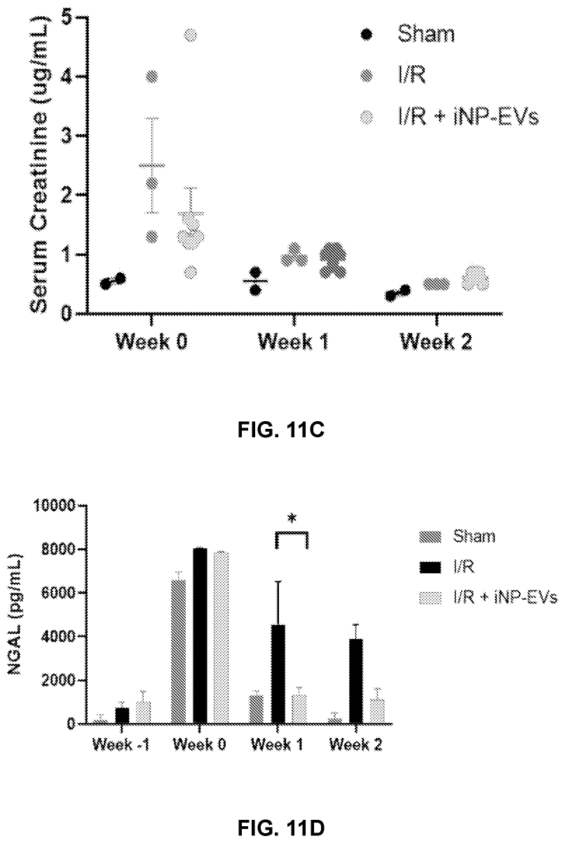

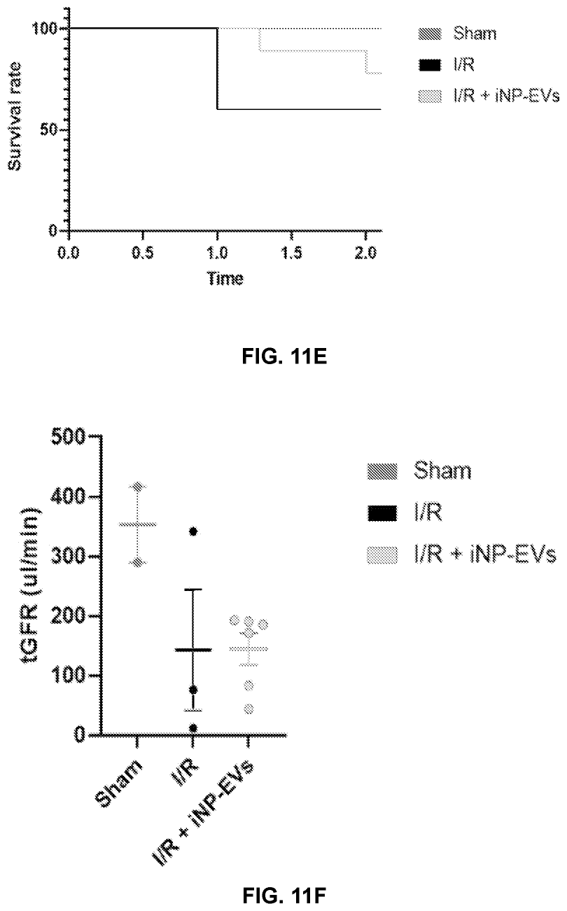

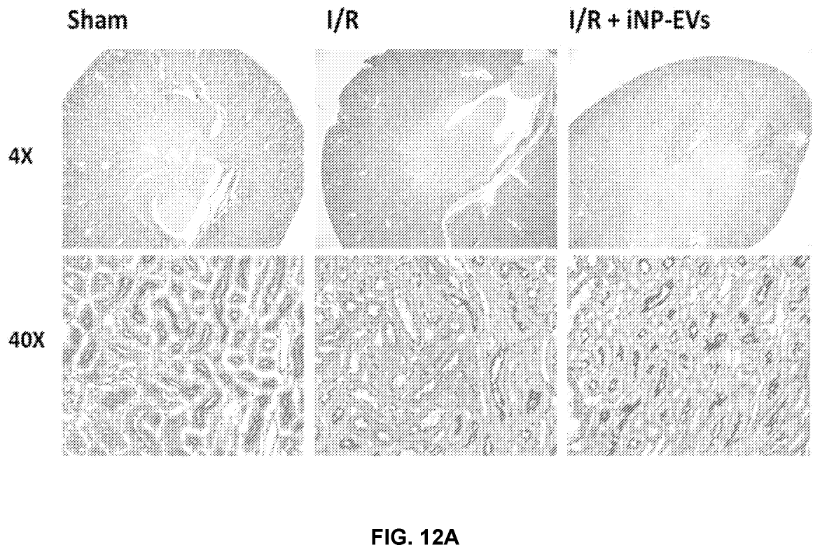

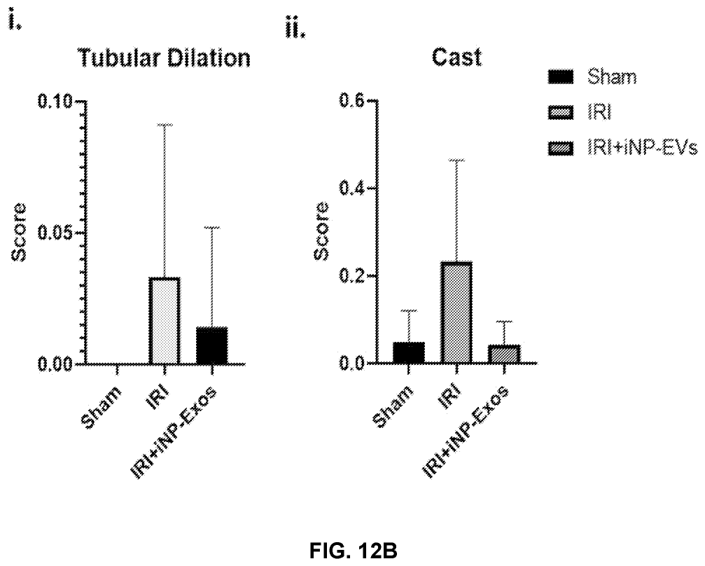

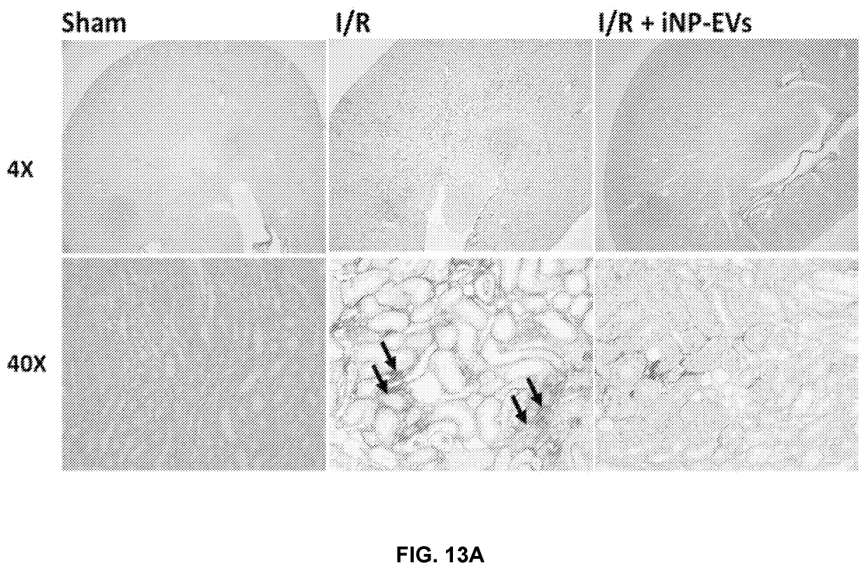

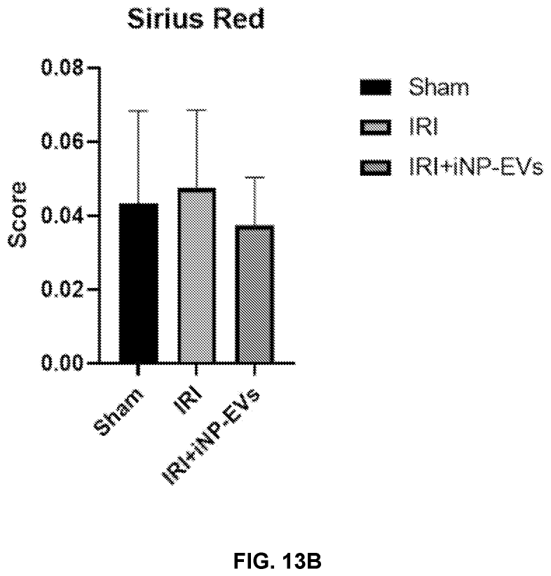

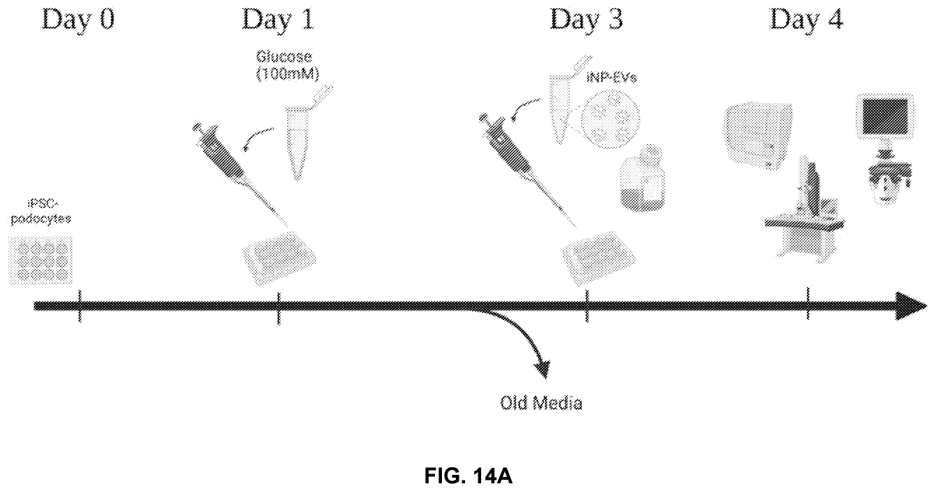

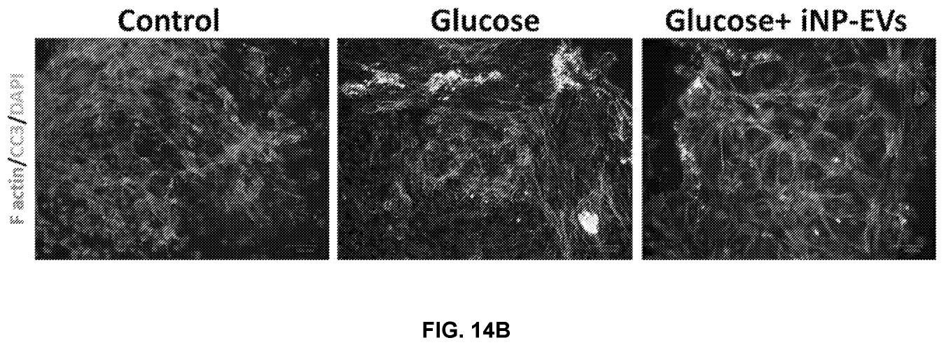

Urine and blood was collected from mice at Week −1 (baseline), Week 0 (I/R), Week 1, Week 2 (FIG. 11A). The levels of serum BUN and sCr were increased in both the control I/R group and I/R+iNP-EVs group 24 hours after surgery, indicating successful injury (FIG. 11B,11C). To evaluate the effects of iNP-EVs on prevention of distal tubule injury in an I/R mouse model of AKI, ELISA of the kidney injury biomarker NGAL in urine was conducted. NGAL ELISA revealed that that iNP-EVs reduced the injury marker expression compared to the I/R control group (FIG. 11D). The mice started to die within the first week after the surgery. In the I/R group the 2-week survival rate was 60%, whereas in the I/R+iNP-EV group it was 77% (FIG. 11E). Transcutaneous tGFR measurements found that the glomerular filtration rate of mice decreased in both I/R group and I/R+EV groups after surgery (FIG. 11F).

Changes in the Tissue Morphology after Treatment with iNP-EVs.

At least 10 cortical fields of periodic acid-Schiff (PAS)-stained slices were assessed and tubular damage scored in a blinded fashion. Tubular injury was defined as tubular vacuolization, loss of the brush border, tubular dilation, and/or tubular cast formation (FIG. 12A). No significant increase in tubular injury was found when comparing I/R to I/R+iNP-EVs. However, there was a significant difference in the presence of casts between groups. I/R groups had more casts compared to the sham whereas I/R+iNP-EVs group were comparable to sham group (FIG. 12C). There was reduction in tubular dilation in the I/R+iNP-EVs group compared to I/R group but was not significant (FIG. 12B). Differences were discovered in the Sirius Red stained sections regarding fibrosis. The fibrosis in the group given INP-EVs was trending lower, with fewer areas of abundant collagen deposition apparent in these mice following AKI injury as compared to the control group given saline (FIG. 13A, 13B).

### INP-EVs May Improve Cytoskeletal Architecture in an In Vitro Diabetic Kidney Disease Model.

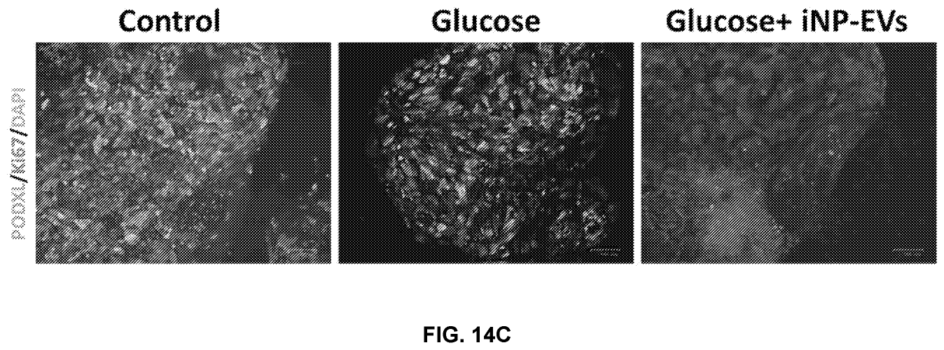

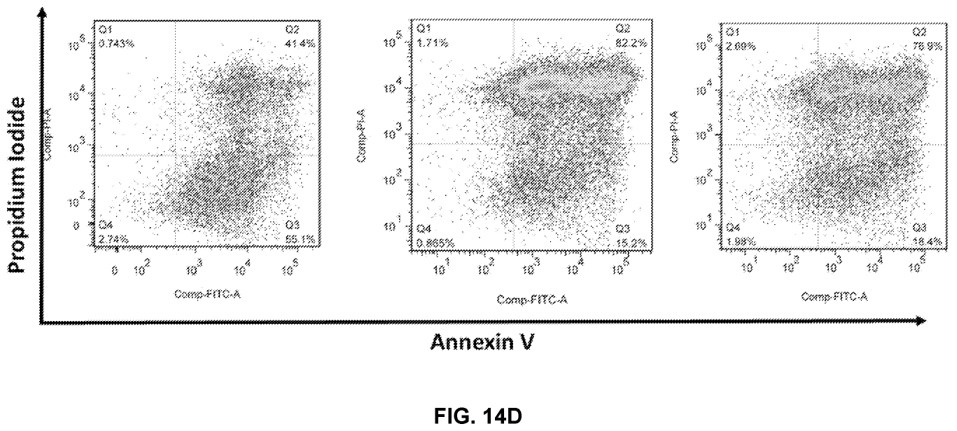

Diabetic kidney disease (DKD) models were have previously published using IPSC-derived podocytes (Grange, C. & Bussolati, B. Nature Reviews Nephrology 2022 18:499-513; Bejoy, J., et al. Stem Cell Research & Therapy 2022 13:355). this high-glucose model was used to investigate the regenerative effect of INP-EVs in DKD (FIG. 14A). Treating podocytes with glucose resulted in out-of-order and intertwined actin filament organization in high glucose treated groups which was reduced by the INP-EVs treatment (FIG. 14B). Further analysis using co-staining with cleaved Caspsase 3 (CC3) showed reduced CC3+ cells in cell receiving INP-EVs in comparison with the high glucose alone group (FIG. 14B). Proliferation analysis using Ki-67 staining indicated the presence of more Ki-67+ cells in the high glucose INP-EVs group in comparison with the high glucose alone group (FIG. 14C). The quantification of apoptosis using Annexin V flow analysis showed a possible trend toward a decrease in apoptosis (FIG. 14D).

### Discussion

EVs have potential as a next-generation regenerative treatment for many diseases because they are able to induce paracrine signaling with more feasible production, storage, distribution, and administration than products consisting of live cells (Grange, C., et al. Stem Cell Investig. 2017 4:90). INPs were derived from the HK-2 clonally-derived cell line HK2-C8, containing an integrated piggyBac transposon carrying the transcription factors SNAI2, SIX1, and EYA1 for doxycycline-inducible cellular reprogramming. INPs are similar to the nephron progenitors of the cap mesenchyme in the developing embryonic kidney. It is unknown to what extent EVs from nephron progenitors influence kidney development and growth. Because EVs from other stem and progenitor cell types improve kidney function and INPs represent a promising cell type for kidney regeneration with kidney-specific regeneration pathways, it was hypothesized that INP-EVs could be an effective treatment for AKI.

During EV isolation, we discovered that EVs could be obtained from INP conditioned media at concentrations that were higher than expected. Therefore, it was possible to harvest sufficient INP-EVs for these experiments from HK-2 Clone 8 INP cells grown on 10 cm plates without the need for 3D culture in bioreactors. Of note, NPs may be successfully grown in 3D culture systems, suggesting the possibility of a relatively simple scale-up process into such bioreactors (Li, Z., et al. Cell Stem Cell 2016 19:516-529). The INPs express nephron progenitor markers SIX2 and OSR1 were previously shown to be at relatively high mRNA levels by Day 6 of reprogramming, with an increase in NP marker SALL1 by Day 8 (Vanslambrouck, J. M. et al. Kidney Int. 2019 95 (5): 1153-1166). Therefore, the INP-EVs were harvested between Days 6-8. At Day 8, 37.6% and 59.7% of the HK2-INPs were expressing NP markers SIX2 and CITED1, indicating successful reprogramming. The HK2-INPs were previously shown to highly express RNA for the off-target stromal marker FOXD1 by Day 10 (Vanslambrouck, J. M. et al. Kidney Int. 2019 95 (5): 1153-1166), so it was decided not to continue harvesting media at later timepoints.

The simplest in vitro models of AKI are immortalized tubule cell lines treated with toxicants. In the nephrotoxicity-induced AKI model, cisplatin was administered to HK2 cells to induce cell death. A proliferative and anti-apoptotic effect of the INP-EVs was observed. In the sepsis-associated AKI model using cell-free hemoglobin treatment of HK2 cells, improved cell viability was observed. When used in a cisplatin-treated kidney organoid model, in addition to a proliferative effect and reduction of injury, INP-EVs reduced the release of inflammatory cytokines.

In mice provided with INP-EVs to support their recovery following renal pedicle clamping I/R AKI, BUN and sCr was measured within 48 hours of EV treatment and found a slight reduction. There was also a reduction of kidney injury marker Kim-1 in the I/R+INP-EVs group compared to I/R alone group. H&E staining as well as PAS staining also showed improved tissue morphology in the I/R+INP-EVs group. Analysis of kidney function in long term function was analyzed at 2 weeks after the INP-EV injection. There was no improvement of BUN or sCr between the INP-EV treated and untreated groups at 1 or 2 weeks post-EV administration. This is likely due to the fast recovery of all of the mice by Week 1 following injury. Urinary NGAL was tested showing a significant decrease in this marker of distal tubule injury at Week 1 and Week 2 following I/R in mice that received INP-EVs vs control mice. The NGAL levels in mice receiving INP-EVs 24 h post-AKI were approximately equal to sham surgery mice that did not receive AKI. This suggests that the administration of the INP-EVs had downstream protective effects on the distal tubule that could be detected at later timepoints. Kidneys were harvested at 2 weeks following injury to examine tubular necrosis and fibrosis. There was no difference in blinded renal tubular necrosis scores from PAS staining between the groups for kidneys harvested at 2 weeks following injury. However, there were improvements in fibrosis between the two groups of mice with many mice in the INP-EV treated group appearing similar to sham controls.

Taken together, these results suggest that while the INP-EV administration one day following injury had protective effects on the distal tubule as suggested by a significant decrease in urinary NGAL. It is not surprising that administration at 24 h was not able to fully reverse the tubular damage that results following AKI, but the histological analysis showed promising trends that may be supported by further studies. This study suggests that further investigation into the timing of INP-EV dosage could reveal a greater protective effect on the proximal tubule if administration occurred earlier or with repeated doses. It is promising that administration at 24 h had significant, protective effects on distal tubule and possibly on the renal cells that deposit collagen leading to fibrosis. Such cell types may have maladaptive responses subsequent to the initial proximal tubule necrosis. We chose the 24 h timepoint because it is a clinically realistic timepoint that sets the bar high for investigation of a regenerative therapeutic.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising nephron progenitor extracellular vesicles (EVs), wherein the EVs are produced by a method comprising:
(a) engineering donor cells to express exogenous SNAI2, EYA1, and SIX1, thereby producing induced nephron progenitor (iNP) cells, or obtaining nephron progenitor-like cells from less differentiated cell types including pluripotent stem cells such as induced pluripotent stem cells or embryonic stem cells;
(b) culturing the iNP or NPC cells in a culture medium, thereby producing a conditioned medium; and
(c) collecting and purifying nephron progenitor EVs from the conditioned medium.

2. The composition of claim 1, wherein the EVs have an average diameter of 50 nm to 150 nm.

3. The composition of claim 1, wherein the EVs are loaded with a therapeutic agent.

4. The composition of claim 1, further comprising a pharmaceutically acceptable carrier.

5. A method for treating or preventing acute kidney injury in a subject, comprising administering to the subject an effective amount of the composition of claim 1.

6. The method of claim 5, wherein the donor cells are autologous.

7. The method of claim 5, wherein the donor cells are allogeneic.

8. The method of claim 5, wherein the composition is administered after onset of the injury.

9. The method of claim 5, wherein the composition is administered prophylactically to a subject at risk for acute kidney injury.

10. The method of claim 5, wherein the subject is the recipient of a kidney transplant.

11. The method of claim 5, wherein the composition is administered to a subject undergoing dialysis.

12. A method comprising treating a donor kidney with effective amount of the composition of claim 1 prior to transplantation into a subject in need thereof.


SEQUENCE LISTING (3 sequences)

SEQ ID NO: 1 - DNA, 807 bp, Homo sapiens
  source 1..807: mol_type = genomic DNA
atgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacactga

SEQ ID NO: 2 - DNA, 855 bp, Homo sapiens
  source 1..855: mol_type = genomic DNA
atgtcgatgctgccgtcgtttggctttacgcaggagcaagtggcgtgcgtgtgcgaggttctgcagcaaggcggaaacctggagcgcctgggcaggttcctgtggtcactgcccgcctgcgaccacctgcacaagaacgagagcgtactcaaggccaaggcggtggtcgccttccaccgcggcaacttccgtgagctctacaagatcctggagagccaccagttctcgcctcacaaccaccccaaactgcagcaactgtggctgaaggcgcattacgtggaggccgagaagctgcgcggccgacccctgggcgccgtgggcaaatatcgggtgcgccgaaaatttccactgccgcgcaccatctgggacggcgaggagaccagctactgcttcaaggagaagtcgaggggtgtcctgcgggagtggtacgcgcacaatccctacccatcgccgcgtgagaagcgggagctggccgaggccaccggcctcaccaccacccaggtcagcaactggtttaagaaccggaggcaaagagaccgggccgcggaggccaaggaaagggagaacaccgaaaacaataactcctcctccaacaagcagaaccaactctctcctctggaagggggcaagccgctcatgtccagctcagaagaggaattctcacctccccaaagtccagaccagaactcggtccttctgctgcagggcaatatgggccacgccaggagctcaaactattctctcccgggcttaacagcctcgcagcccagtcacggcctgcagacccaccagcatcagctccaagactctctgctcggccccctcacctccagtctggtggacttggggtcctaa

SEQ ID NO: 3 - DNA, 1779 bp, Homo sapiens
  source 1..1779: mol_type = genomic DNA
atggaaatgcaggatctaaccagcccgcatagccgtctgagtggtagtagtgaatcccccagtggccccaaactcggtaactctcatataaatagtaattccatgactcccaatggcaccgaagttaaaacagagccaatgagcagcagtgaaacagcttcaacgacagccgacgggtctttaaacaatttctcaggttcagcaattgggagcagtagtttcagcccacgaccaactcaccagttctctccaccacagatttacccttccaacagaccatacccacatattctccctaccccttcctcacaaactatggctgcatatgggcaaacacagtttaccacaggaatgcaacaagctacagcctatgccacgtacccacagccaggacagccgtacggcatttcctcatatggtgcattgtgggcaggcatcaagactgaaggtggattgtcacagtctcagtcacctggacagacaggatttctcagctatggcacaagcttcagtacccctcaacctggacaggcaccatacagctaccagatgcaaggtagcagttttacaacatcatcaggaatatatacaggaaataattcactcacaaattcctctggatttaatagttcacagcaggactatccgtcttatcccagttttggccagggtcagtacgcacagtattataacagctcaccgtatccagcacattatatgaccagcagcaacaccagcccaacgacaccatccaccaatgccacttaccagcttcaagaaccgccatctggcatcaccagccaagcagttacagatcccacagcagagtacagcacaatccacagcccatcaacacccattaaagattcagattctgatcgattgcgtcgaggttcagatgggaaatcacgtggacggggccgaagaaacaataatccttcacctcccccagattctgatcttgagagagtgttcatctgggacttggatgagacaatcattgttttccactccttgcttactgggtcctacgccaacagatatgggagggatccacccacttcagtttcccttggactgcgaatggaagaaatgattttcaacttggcagacacacatttattttttaatgacttagaagaatgtgaccaagtccatatagatgatgtttcttcagatgataacggacaggacctaagcacatataactttggaacagatggctttcctgctgcagcaaccagtgctaacttatgtttggcaactggtgtacggggcggtgtggactggatgagaaagttggccttccgctacagacgggtaaaagagatctacaacacctacaaaaataatgttggaggtctgcttggtccagctaagagggaagcctggctgcagttgagggccgaaattgaagccctgaccgactcctggttgacactggccctgaaagcactctcgctcattcactcccggacaaactgtgtgaatattttagtaacaactactcagctcatcccagcattggcgaaagtcctgctgtatgggttaggaattgtatttccaatagaaaatatttacagtgcaactaaaataggaaaagaaagctgttttgagagaataattcaaaggtttggaagaaaagtggtgtatgttgttataggagatggtgtagaagaagaacaaggagcaaaaaagcacgcgatgcccttctggaggatctccagccactcggacctcatggccctgcaccatgccttggaactggagtacctgtaa